(19) United States
(12) Patent Application Publication
(10) Pub. No.: US 2025/0090317 A1
(43) Pub. Date: Mar. 20, 2025

(54) SKIRT ASSEMBLIES FOR PROSTHETIC VALVES

(71) Applicant: EDWARDS LIFESCIENCES CORPORATION, Irvine, CA (US)

(72) Inventors: Nikolai Gurovich, Hadera (IL); Michael Bukin, Pardes Hanna (IL); Eran Grosu, Kfar-Saba (IL); Ilan Leshecz, Hadera (IL)

(73) Assignee: EDWARDS LIFESCIENCES CORPORATION, Irvine, CA (US)

(21) Appl. No.: 18/967,404

(22) Filed: Dec. 3, 2024

Related U.S. Application Data
(63) Continuation of application No. PCT/US2023/025491, filed on Jun. 16, 2023.
(60) Provisional application No. 63/355,257, filed on Jun. 24, 2022.

(51) Int. Cl. A61F 2/24

(52) U.S. Cl. CPC A61F 2/2418; A61F 2/2436; A61F 2210/0014; A61F 2220/0025; A61F 2250/0069

(57) ABSTRACT

A prosthetic heart valve includes a valve frame comprising an outflow end and an inflow end, a plurality of leaflets disposed within and coupled to the valve frame, and a skirt assembly mounted to an outer surface of the valve frame. The skirt assembly includes a sealing layer and a skirt frame including a plurality of interconnected struts, wherein the skirt frame has a plurality of inflow apices and outflow apices, wherein selected ones of the inflow apices are fixed to the valve frame and selected ones of the outflow apices are fixed to the valve frame, wherein one or more of the struts are configured to flex in an outward radial direction when the valve frame is in a radially expanded state to cause the sealing layer to protrude outwardly from the valve frame.

### CROSS-REFERENCE TO RELATED APPLICATIONS

This application is a continuation of PCT Application No. PCT/US2023/025491, filed Jun. 16, 2023, which claims the benefit of U.S. Provisional Application No. 63/355,257, filed Jun. 24, 2022, both of which are incorporated by reference herein.

### FIELD

The present disclosure relates to skirt assemblies for prosthetic valves and systems and methods for skirt assemblies including bulging features to reduce paravalvular leakage.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Percutaneous prosthetic valves (also known as transcatheter heart valves) typically have an outer skirt that extends around the outer surface of the frame of the prosthetic valve. When the prosthetic valve is expanded within a native heart valve, the outer skirt contacts tissue of the surrounding native valve, thereby establishing a seal between the prosthetic valve and the surrounding tissue that prevents or reduces paravalvular leakage. Depending on the patient's anatomy, the native valve can have an irregular shape, such as due to the presence of calcium nodules, which can prevent the outer skirt from fully sealing against the surrounding tissue.

Accordingly, a need exists for improved skirt assemblies for preventing or minimizing paravalvular leakage.

### SUMMARY

Described herein are prosthetic heart valves, delivery apparatuses, and methods for implanting prosthetic heart valves. The disclosed prosthetic heart valves can, for example, include skirt assemblies that extend around the outer surface of a frame of the prosthetic valve and bulge or flex away from the frame in a predetermined manner. The disclosed skirt assemblies can help to ensure that the prosthetic heart valve establishes a full seal against the native valve, such that paravalvular leakage is prevented or minimized. As such, the devices and methods disclosed herein can, among other things, overcome one or more of the deficiencies of typical delivery apparatuses for mechanically expandable prosthetic valves.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a skirt assembly configured to reduce paravalvular leakage.

In some examples, a prosthetic heart valve can comprise a skirt assembly comprising a sealing layer and a skirt frame, wherein the skirt frame is configured to flex in an outward radial direction when the prosthetic heart valve is in a radially expanded state.

In some examples, a prosthetic heart valve can comprise a valve frame and a skirt assembly, the skirt assembly comprising a sealing layer and a skirt frame, wherein the skirt assembly is configured to flex radially outwardly relative to the valve frame upon radial expansion of the prosthetic heart valve.

In some examples, the skirt frame comprises a plurality of fixed apices that are fixed to the valve frame and a plurality of free apices configured to flex radially outwardly relative to the valve frame.

In some examples, the skirt frame comprises a plurality of angled struts and a plurality of axial struts.

In some examples, a prosthetic heart valve comprises a valve frame comprising an outflow end and an inflow end, wherein the valve frame is radially expandable from a radially compressed state to a radially expanded state, a plurality of leaflets disposed within and coupled to the valve frame, and a skirt assembly mounted to an outer surface of the valve frame, wherein the skirt assembly comprises a sealing layer and a skirt frame including a plurality of interconnected struts, wherein the skirt frame has a plurality of inflow apices and outflow apices, wherein selected ones of the inflow apices are fixed to the valve frame and selected ones of the outflow apices are fixed to the valve frame, wherein one or more of the struts are configured to flex in an outward radial direction when the valve frame is in the radially expanded state to cause the sealing layer to protrude outwardly from the valve frame.

In some examples, a prosthetic heart valve comprises a valve frame being radially expandable and compressible between a radially compressed state and a radially expanded state, a plurality of leaflets disposed within and coupled to the valve frame, and a skirt assembly mounted to an outer surface of the valve frame, wherein the skirt assembly comprises a scaling layer and a skirt frame, wherein the skirt frame comprises a plurality of interconnected struts forming at least one row of cells, wherein radially expanding the valve frame from the radially compressed state to the radially expanded state results in the skirt assembly flexing radially outwardly relative to the valve frame.

In some examples, a prosthetic heart valve comprises a valve frame comprising an outflow end and an inflow end, wherein the valve frame is radially expandable from a radially compressed state to a radially expanded state, a plurality of leaflets disposed within and coupled to the valve frame, and a sealing assembly mounted to an outer surface of the valve frame, the sealing assembly comprising a sealing member and a sealing frame, wherein the sealing frame comprises a shape-memory material, wherein the sealing frame is in a radially compressed state when the valve frame is in the radially compressed state, wherein the compressed state of the sealing frame is a shape-memory state, and wherein radially expansion of the valve frame results in deformation of the sealing frame from the shape-memory state to a deformed state.

In some examples, a delivery apparatus comprises a delivery device; and a prosthetic valve releasably coupled to the delivery device, the prosthetic valve comprising a valve frame that is expandable between a radially compressed state and a radially expanded state, a valvular structure mounted within the valve frame, and a sealing assembly mounted to an outer surface of the valve frame, wherein the sealing assembly comprises a sealing layer and a sealing frame including a plurality of interconnected struts, wherein the sealing frame has a plurality of inflow apices and outflow apices, wherein selected ones of the inflow apices are fixed to the sealing frame and selected ones of the outflow apices are fixed to the scaling frame, wherein one or more of the struts are configured to flex in an outward radial direction when the valve frame is in the radially expanded state to cause the sealing layer to protrude outwardly from the sealing frame.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6 is a perspective view of the prosthetic valve of FIG. 1A with a skirt assembly disposed around the frame, according to one example.

FIG. 7 is a perspective view of the prosthetic valve of FIG. 6 with the sealing layer of the skirt assembly removed for purposes of illustration.

FIG. 8 is a perspective view of the valve frame and the skirt frame of the prosthetic valve of FIG. 6 shown in a radially expanded state.

FIG. 9 is a perspective view of the valve frame and the skirt frame of FIG. 8 shown in a radially compressed state.

FIG. 10 is an enlarged, partial side view of the valve frame and the skirt frame of FIG. 8 in a radially expanded state.

FIG. 11 is a side view of a prosthetic heart valve, according to one example.

FIG. 12 is a side view of a frame of the prosthetic heart valve of FIG. 11.

FIG. 13 is a side view of a portion of the frame of FIG. 12, showing the portion of the frame in a straightened (non-annular) state.

FIG. 14 is a side view of an exemplary delivery apparatus configured to deliver and implant a radially expandable prosthetic heart valve at an implantation site.

FIG. 15 is a perspective view of the frame of FIG. 12 with a skirt assembly disposed around the frame, according to one example.

FIG. 16 is a perspective view of a skirt frame of the skirt assembly of FIG. 15.

FIG. 17 is a perspective view of the frame of FIG. 12 with a skirt assembly disposed around the frame, according to another example.

FIG. 18 is a perspective view of the frame of FIG. 12 with a skirt assembly disposed around the frame, according to another example.

FIG. 19 is a partial side view of a skirt frame coupled to a cell of a valve frame in a crimped state.

FIG. 20 is a perspective view of the frame of FIG. 12 with a skirt assembly disposed around the frame, according to another example.

FIG. 21 is a side view of a skirt frame of the skirt assembly of FIG. 20 in a flattened state.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Described herein are various examples of outer skirt assemblies for prosthetic heart valves that can be disposed around an outer surface of the prosthetic heart valve and that are configured to form a seal against native tissue upon implantation of the prosthetic heart valve, thereby reducing paravalvular leakage (PVL) past the prosthetic heart valve when expanded against the native anatomy. The outer skirt assemblies described herein can include a sealing member (for example, a skirt) that is coupled to a skirt frame. The skirt frame can be coupled to the outer surface of the prosthetic heart valve and be configured to expand radially away from the valve in a controlled manner, such that the desired profile, size, and locations of portions of the skirt frame that bulge or flex away from the prosthetic heart valve can be predetermined. As such, the bulging portions of the skirt assemblies disclosed herein can form an improved seal against native tissue upon implantation of the prosthetic heart valve, and thus reduce PVL past the prosthetic heart valve.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

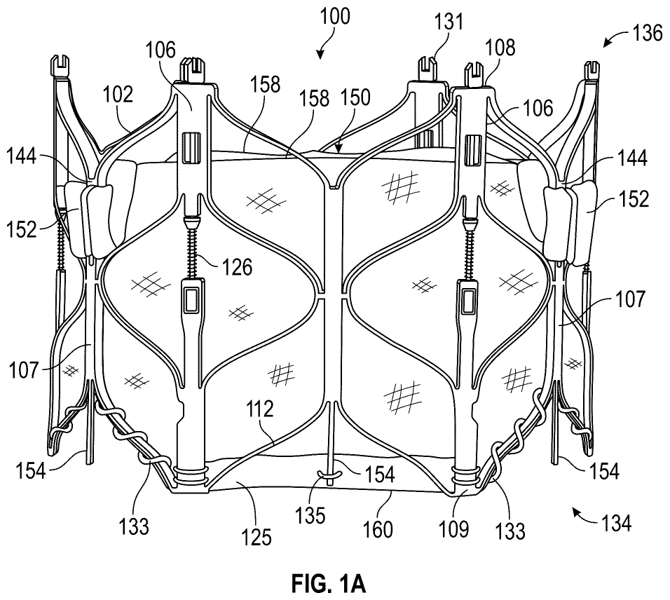

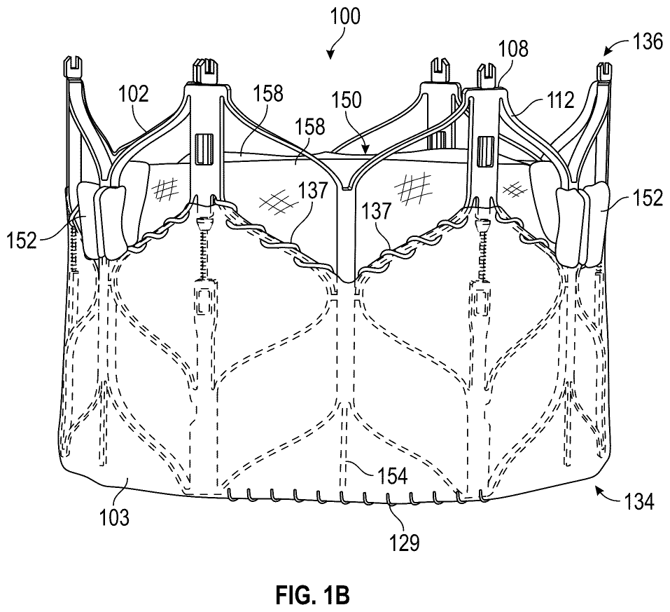

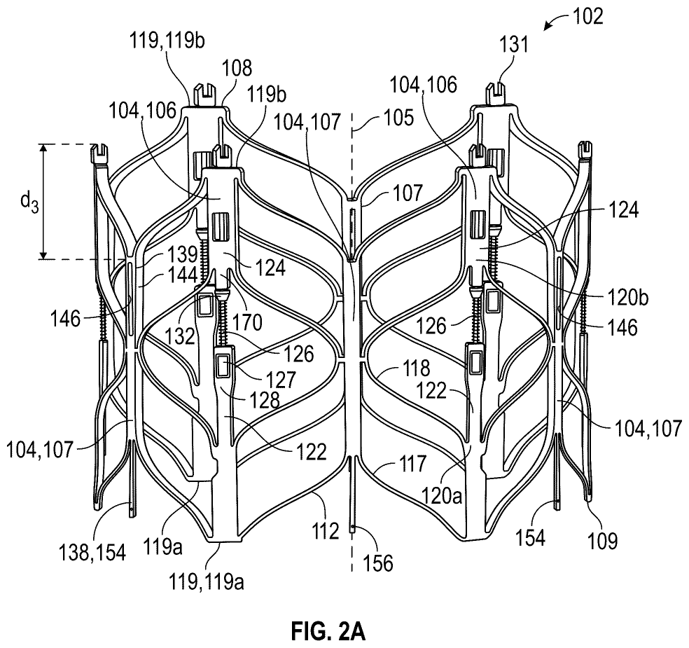

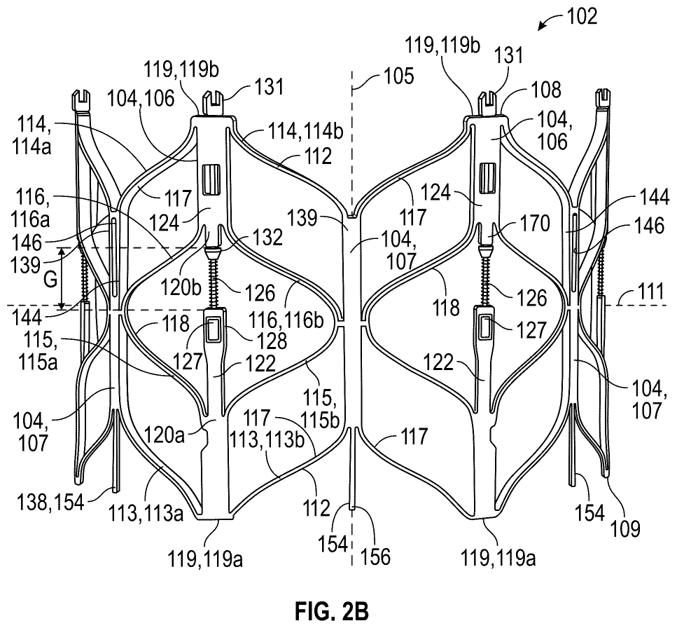

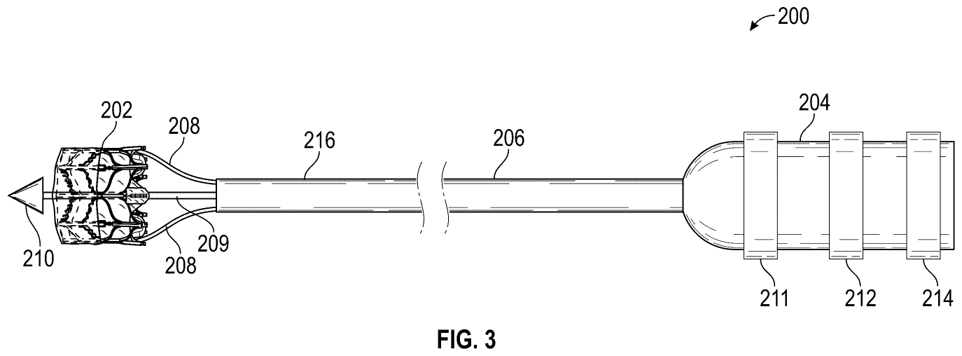

FIGS. 1A-2B illustrate an example prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame-specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

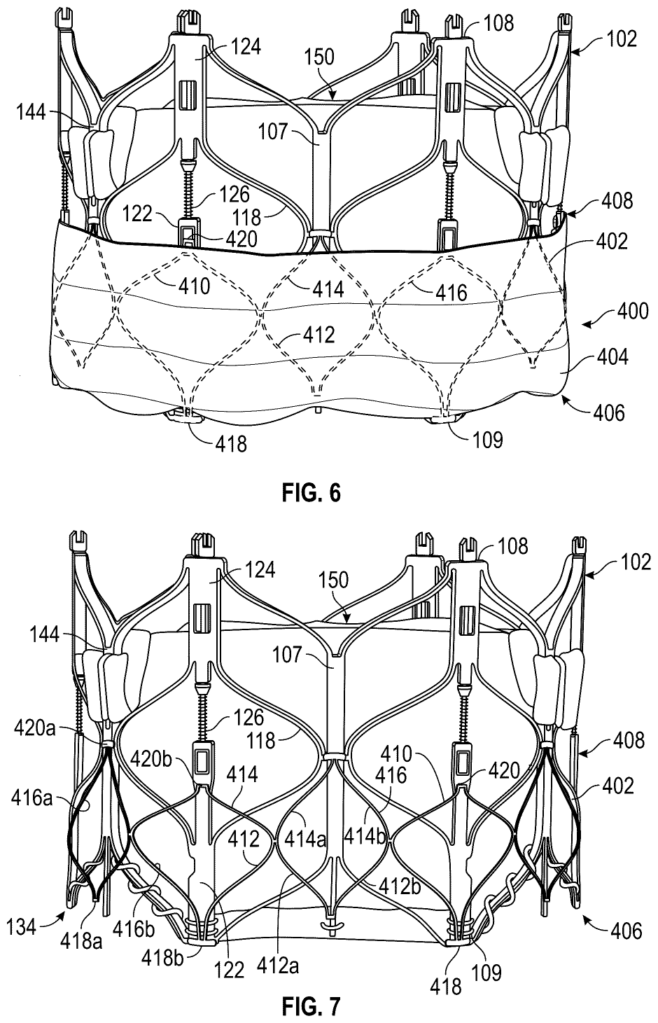

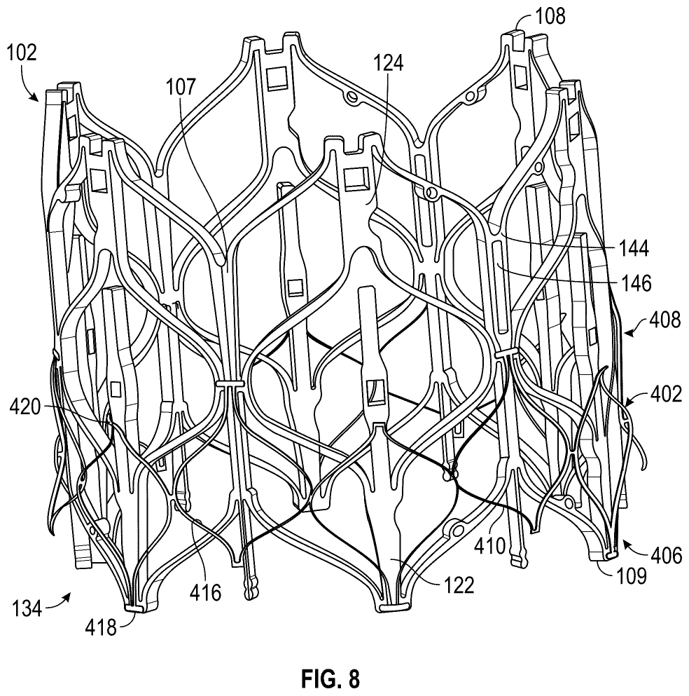

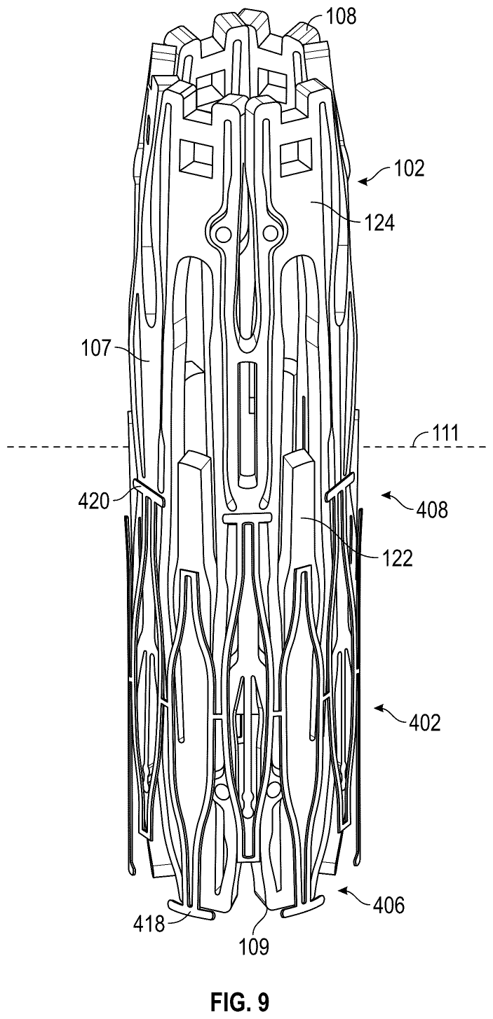

In some examples, a skirt assembly for a prosthetic heart valve, such as the skirt assembly depicted in FIGS. 6-10, can be configured with a sealing member (or scaling layer) coupled to a skirt frame that includes multiple rows of angled struts that define a plurality of cells. The cells can have fixed apices that are fixedly coupled to a valve frame of the prosthetic heart valve and free apices that are movable relative to the valve frame. The free portions of the skirt frame (portions unattached to the main frame of the prosthetic valve) can be configured to protrude radially outward from the valve frame of prosthetic heart valve when the prosthetic heart valve is radially expanded (as shown in FIGS. 6-8 and 10). The skirt frame can be relatively thin, such that the skirt assembly does not significantly add to the overall outer diameter of the prosthetic heart valve when in a crimped state (as shown in FIG. 9).

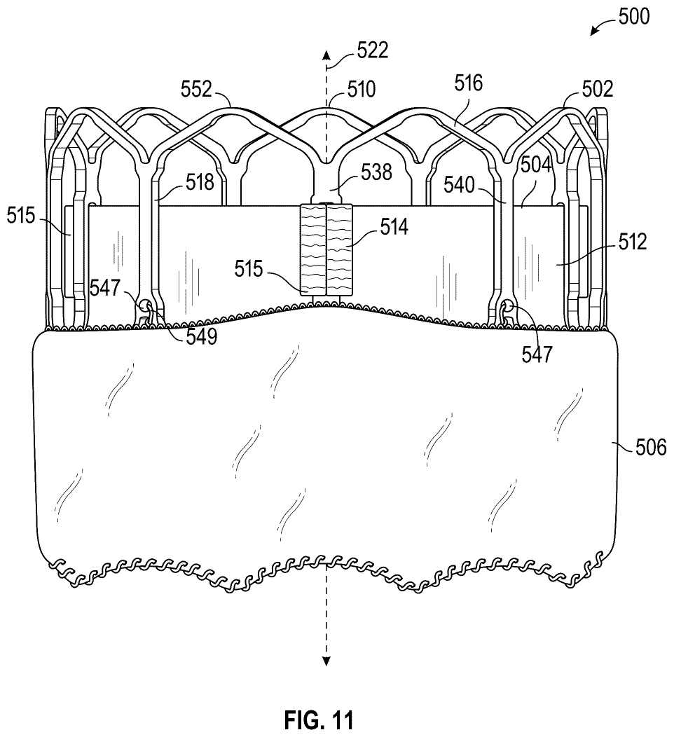

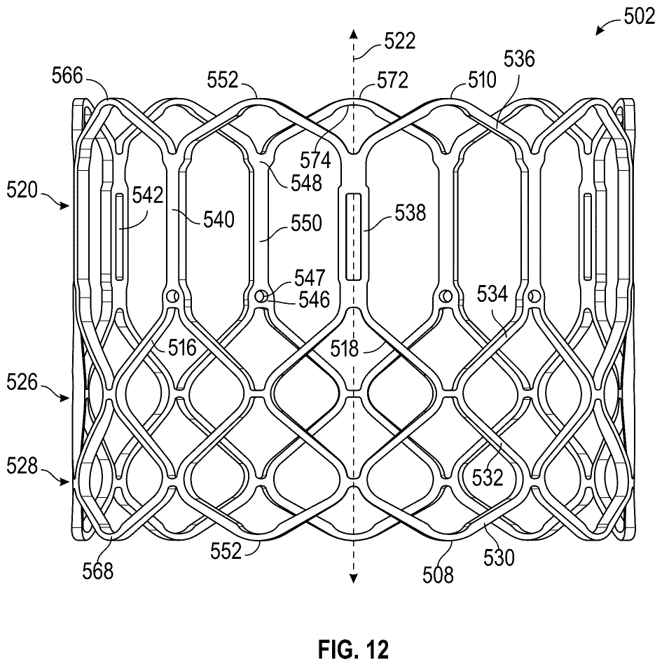

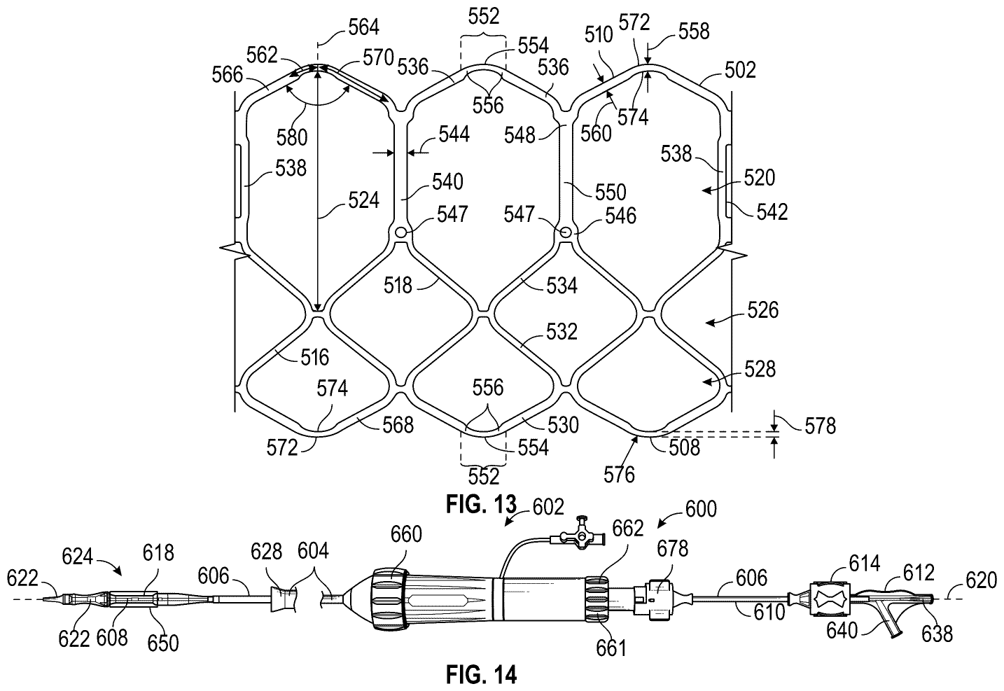

FIG. 11 illustrates an exemplary prosthetic device (for example, prosthetic heart valve) comprising a frame, leaflets secured on an inside of the frame, and an outer skirt disposed around an outer surface of the frame. In some examples, the frame can comprise a plurality of interconnected and angled struts and apex regions that extend and/or curve between the angled struts at an inflow end and outflow end of the frame, as shown in FIGS. 12 and 13. The prosthetic device can be advanced through a patient's vasculature, such as to a native heart valve, by a delivery apparatus, such as the exemplary delivery apparatus shown in FIG. 14.

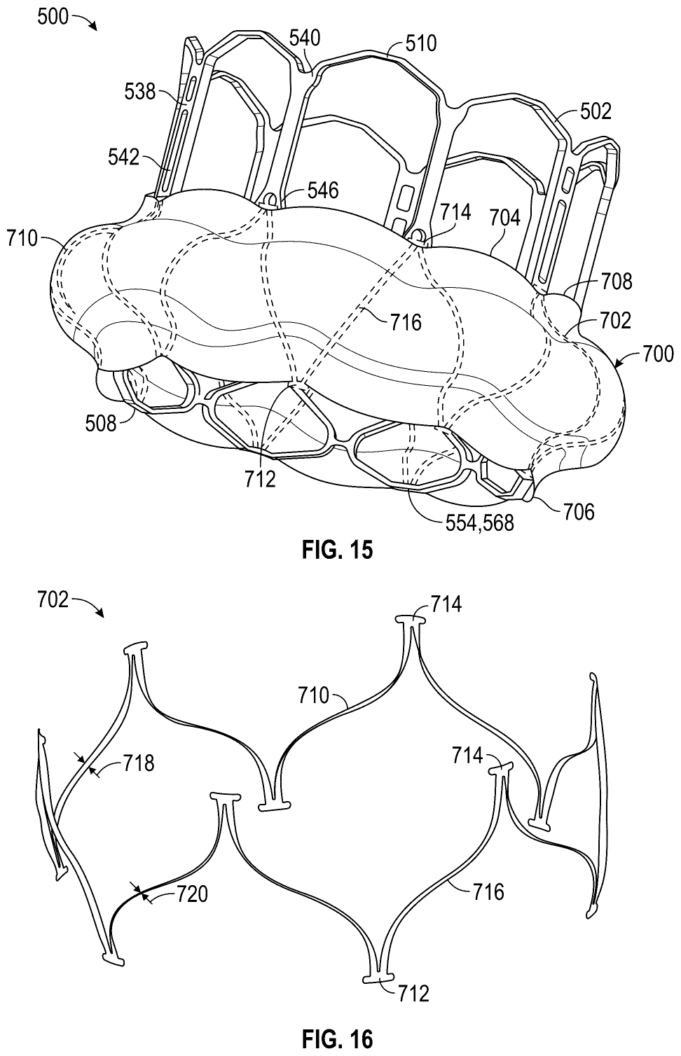

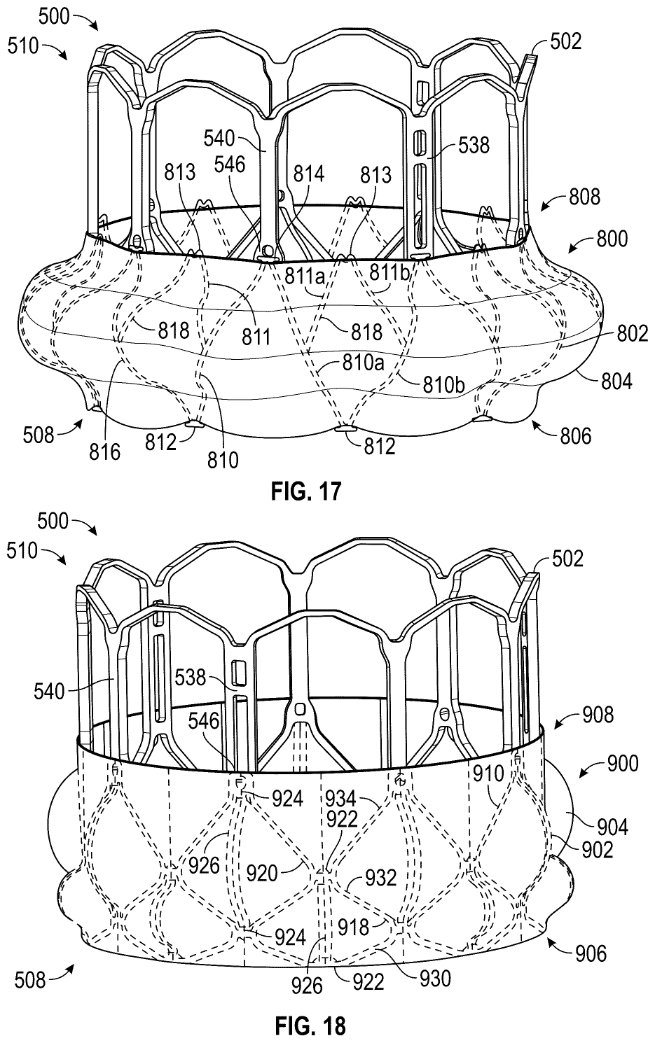

In some examples, as depicted in FIGS. 15-16, the skirt assembly can include a skirt frame that includes only one row of angled struts and each apex defined by the struts are coupled to the valve frame. The intermediate portions of the struts are not coupled to the valve frame and are configured to bulge or flex away from the valve frame when the prosthetic heart valve is radially expanded. In some examples, as depicted in FIG. 17, the skirt assembly can include two rows of angled struts that define a row of circumferentially spaced apart cells. In some examples, as depicted in FIGS. 18-22, a skirt frame can include three rows of struts that are configured to bulge or flex away from the valve frame in different manners when the prosthetic heart valve is radially expanded. For example, a skirt frame can include axially extending struts that are permitted to bulge away from the valve frame when the prosthetic heart valve is radially expanded (as shown in FIG. 18).

### Examples of the Disclosed Technology

FIGS. 1A-2B show a prosthetic valve 100, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded state. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In other examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate (PET) fabric), non-textile synthetic materials (for examples, made from any of various polymers) or natural tissue (for example, pericardial tissue). Other fabric or polymeric materials that can be used to form the skirts include, without limitation, expanded polytetrafluoroethylene (ePTFE), polytetrafluoroethylene (PTFE), thermoplastic polyurethane (TPU), ultra-high molecular weight polyethylene (UHMWPE), polyether ether ketone (PEEK), polyethylene (PE), etc. Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises an inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In other examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in other examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline 111 of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in other examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In other examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In other examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed state to an expanded state, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In other examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in other examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in other examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the frame 102 can be formed from a shape-memory material (for example, Nitinol). When the frame 102 is formed from a shape-memory material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule and can self-expand to a radially expanded state. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 3, it illustrates an example of a delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In other examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In other examples, the first knob 211 can be actuated by sliding or moving the first knob 211 axially, such as pulling and/or pushing the knob. In other examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In other examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

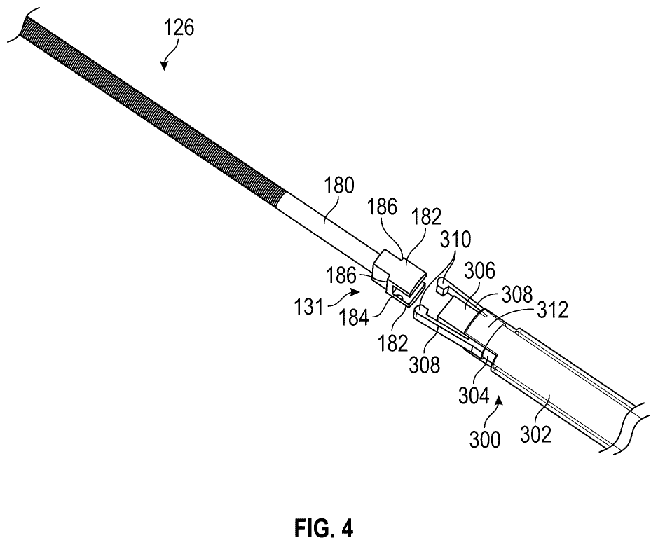

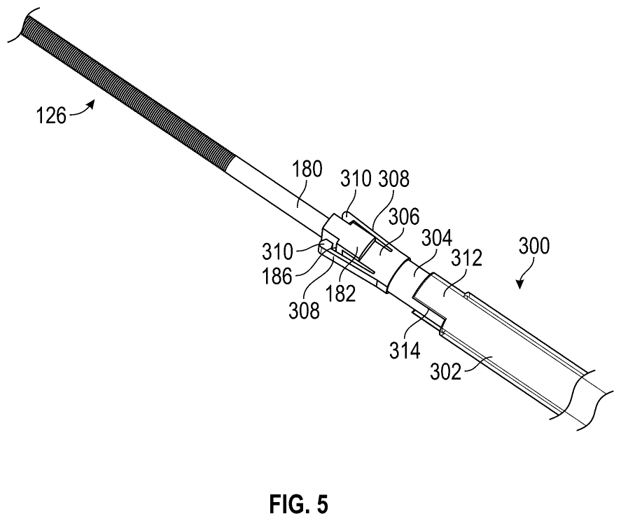

Referring to FIGS. 4-5, they illustrate how each of the threaded rods 126 of the prosthetic device 100 can be removably coupled to an actuator assembly 300 (for example, actuator assemblies 208) of a delivery apparatus (for example, delivery apparatus 200), according to one example. Specifically, FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In other examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the driver 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in other examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated example is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

In some examples, the outer skirt 103 of the prosthetic heart valve 100 can be replaced with a skirt assembly (for example, any of the skirt assemblies described herein). For example, referring to FIGS. 6-10, the prosthetic valve 100 may include a skirt assembly 400 (also referred to as a “sealing assembly 400”) mounted around the frame 102 (also referred to as a “valve frame 102”). The skirt assembly 400 can function as a scaling mechanism for the prosthetic valve 100 by sealing against the tissue of the native valve annulus and helping to reduce paravalvular leakage past the prosthetic valve 100. For example, as shown in FIG. 6, the skirt assembly 400 may include a skirt frame 402 (also referred to as a sealing frame) and a skirt 404 (also referred to as a sealing member or sealing layer) mounted around an outer surface of the valve frame 102. FIGS. 8-10 show the valve frame 102 and the skirt frame 402 by themselves, while FIG. 7 additionally shows the valvular structure 150 mounted within and to the valve frame 102.

The skirt assembly 400 may include a skirt 404 coupled to the skirt frame 402, as shown in FIG. 6. In some examples, as shown, the skirt 404 can be mounted around an outer surface of the skirt frame 402. In other examples, the skirt 404 can be positioned radially inward of the skirt frame 402 and can be mounted to an inner surface of the skirt frame 402 (for example, with sutures, etc.). The skirt 404 can be made from any of various materials, including any of those described above in connection for the inner and outer skirts of the prosthetic valve of FIGS. 1A and 1B.

In other examples, the skirt 404 can have an outer layer positioned on the outer surface of the skirt frame 402 and an inner layer positioned on the inner surface of the skirt frame 402 such that the skirt frame 402 is disposed between the inner and outer layers. The inner and outer layers can be formed from a single piece of skirt material; for example, the skirt 404 can comprise a single or unitary piece of material that extends along the outer surface of the skirt frame, is folded around an inflow or outflow edge of the skirt frame, and extends along the inner surface of the skirt frame. Alternatively, the inner and outer layers can be separate pieces of material.

In still other examples, the skirt frame 402 can be embedded within the skirt 404 (for example, the skirt 404 can have yarns that are woven around the struts of the skirt frame 402).

The skirt 404 can function as a sealing member for the prosthetic valve 100 by sealing against the tissue of the native valve annulus and helping to reduce paravalvular leakage past the prosthetic valve 100. As will be described in more detail below, the skirt frame 402 can be configured to radially bulge or flex away from the valve frame 102 to improve sealing of the prosthetic valve 100 against the tissue of the native valve annulus and help reduce paravalvular leakage.

The skirt frame 402 can be coupled (for example, fixedly mounted) to an outer surface of the valve frame 102. In some examples, the skirt frame 402 can be welded to the valve frame 102 or adhered to the valve frame with a suitable biocompatible adhesive. In other examples, the skirt frame 402 can be fastened to the valve frame 102, for example, by one or more sutures, mechanism fasteners (such as, pins, screws, or rivets) or some other attachment mechanism, etc.

The skirt frame 402 can be mounted at various locations along the length of the valve frame 102. Moreover, the skirt frame 402 can be mounted to any number of the posts 104 and/or to any number of the struts 112 of the valve frame 102. In some examples, as depicted in FIG. 7 for example, portions of the skirt frame 402 can be mounted to each of the support posts 107 as well as to each of the actuator posts 122. As shown, the skirt frame 402 is positioned closer towards the inflow end portion 134 of the prosthetic valve 100 than the outflow end portion of the prosthetic valve such that the skirt frame is axially offset from the outflow end of the valve frame. In particular, a first (or inflow) end 406 of the skirt frame 402 is coupled to the actuator posts 122 at the inflow end 109 of the valve frame 102 and a second (or outflow) end 408 of the skirt frame 402 is coupled to the support posts 107 (and commissure support structures 144) at an intermediate portion of the valve frame 102 between inflow and outflow ends 109, 108 (for example, closer to the axial midline 111 of the valve frame 102), respectively. For example, as depicted, the second end 408 of the skirt frame 402 is coupled to the valve frame 102 at a location where struts of the second cells 118 connect to the support posts 107 and below the commissure windows 146 (for example, positioned closer to the inflow end 109 than the commissure windows 146, etc.).

In other examples, the skirt frame 402 can be axially offset from the inflow end of the valve frame 102 and can extend from the outflow end of the valve frame to an intermediate portion of the valve frame 102. In still other examples, the skirt frame 402 can be positioned generally along the intermediate portion of the valve frame and axially offset from the inflow and outflow ends of the valve frame 102. In still other examples, the skirt frame 402 can span the entire length of the valve frame 102 from the inflow end of the valve frame to the outflow end of the valve frame.

The skirt frame 402 can include a plurality of circumferentially extending link members or struts 410. As illustrated in FIG. 7, the struts 410 can include a first row of struts 412 at or near the first end 406 of the skirt frame 402 (for example, at or near the inflow end 109 of the prosthetic valve 100) and a second row of struts 414 at or near the second end 408 of the skirt frame 402. The struts 410 can form and/or define a plurality of cells (that is, openings) in the skirt frame 402. The cells can be arranged to form a circumferentially extending row of cells connected side-by-side to each other in the row, as shown. For example, the struts 412 and 414 can at least partially form and/or define a plurality of cells 416 that extend circumferentially around the skirt frame 402. Specifically, each cell 416 can be formed by two struts 412a, 412b of the first row of struts 412 and two struts 414a, 414b of the second row of struts 414. Each cell 416 can have a diamond shape including first (or inflow) apices 418 at or near the first end 406 of the skirt frame 402 and second (or outflow) apices 420 at or near the second end 408 of the skirt frame 402. In some examples, the skirt frame 402 comprises twelve cells 416 extending circumferentially in a row. However, in other examples, the skirt frame 402 can comprise a greater or fewer number of cells 416 in a row.

While two rows of struts 410 are illustrated in the skirt frame 402 of FIGS. 6-10, in other examples, the skirt frame 402 can include a different number of rows of struts 410 (for example, greater or fewer than two rows) and rows of cells (for example, more than one rows of cells, such as two, three or four rows of cells) arranged in similar or different shapes and patterns. For example, the skirt frame 402 can include a single row of struts (FIGS. 15-16), a different pattern of two rows of struts (FIG. 17), three rows of struts (FIGS. 18-21), etc. as will be described in more detail below, as well as any other number of rows of struts arranged in similar or different patterns. In some examples, such as the example depicted in FIGS. 15-16, the struts of skirt frame do not define any cells.

In some examples, as depicted, each cell 416 can have a free apex that is not connected to the valve frame 102 (for example, moveable relative to the valve frame 102, etc.) and a fixed apex that is connected to the valve frame 102 (for example, fixed relative to the valve frame 102, etc.). Specifically, one cell 416a has a free lower apex 418a and a fixed upper apex 420a and another cell 416b has a fixed lower apex 418b and a free upper apex 420b. As one example, the skirt frame 402 can include equal numbers of cells 416a having free lower apices 418a/fixed upper apices 420a and cells 416b having fixed lower apices 418b/free upper apices 420b. In these examples, the cells 416a, 416b can be arranged in an alternating order such that every other lower apex 418 is fixed to the valve frame 102 (for example, fixed lower apex 418b) and every other upper apex 420 is fixed to the valve frame 102 (for example, fixed upper apex 420a), such as in a zig-zag pattern. However, the skirt frame 402 can include different numbers of cells 416a and cells 416b and/or the cells 416a, 416b can be arranged in a non-alternating order, in other examples. In some examples, as depicted, the fixed apices (fixed lower apices 418b and fixed upper apices 420a) have T-bars for connecting the apices to the valve frame 102. For example, a suture can be wrapped around T-bars or similar connection features of the fixed lower apices 418b and the fixed upper apices 420a to connect the apices 418b, 420a to the posts 104 of the valve frame 102.

As introduced above, the valve frame 102 (and therefore the prosthetic valve 100) can be radially compressible and expandable between a radially compressed state (FIG. 9) and a radially expanded state (FIG. 8). The valve frame 102 has an axial length (for example, from the inflow end 109 to the outflow end 108) that can be defined by the actuator posts 122, 124 and the skirt frame 402 has an axial length (for example, from the inflow end 406 to the outflow end 408 of the skirt frame 402) that can be defined by the inflow and outflow apices 418, 420. The valve frame 102 can be radially expanded (for example, by rotating the actuator 126 relative to the first and second actuator posts 122, 124, etc.) from the radially compressed state to the radially expanded state, which foreshortens the axial length of the valve frame 102. Due to portions of the skirt frame 402 being fixed to the valve frame 102, as the valve frame 102 is axially foreshortened, the axial length of the skirt frame 402 is also foreshortened. As such, in the radially expanded state, the axial lengths of the valve frame 102 and the skirt frame 402 are shorter than in the radially compressed state. Stated another way, the valve frame 102 and the skirt frame 402 axially foreshorten during radial expansion of the valve frame 102.

In some examples, the valve frame 102 and the skirt frame 402 can axially foreshorten by different amounts (for example, based on the attachment locations of the skirt frame 402 to the valve frame 102). For example, during radial expansion of the valve frame 102, the support posts 107 remain stationary in an axial direction relative to the axial midline 111 of the prosthetic valve 100, whereas the actuator posts 122, 124 (which define the axial length of the valve frame 102) move relatively closer in an axial direction towards the axial midline 111 of the prosthetic valve 100. The skirt frame 402 is fixed to both relatively stationary (for example, support posts 107) and relatively moveable (for example, actuator posts 122) points on the valve frame 102. In this way, the amount of axial foreshortening of the skirt frame 402 is dependent on the amount that one of the actuator posts (for example, post 122) moves relative to the axial midline 111. In contrast, the amount of axial foreshortening of the valve frame 102 is dependent on the amount that both of the actuator posts (for example, posts 122, 124) move relative to the axial midline 111. As such, the amount of axial foreshortening of the skirt frame 402 can be less than the amount of axial foreshortening of the valve frame 102. In other examples, the skirt frame 402 can be fixed to other attachment locations or points on the valve frame 102 (for example, the skirt frame 402 is fixed to the valve frame 102 at other points that are stationary relative to the axial midline 111 and/or other points that are moveable relative to the axial midline 111), such that the axial foreshortening of the skirt frame 402 and the valve frame 102 can be the same amount or different amounts.

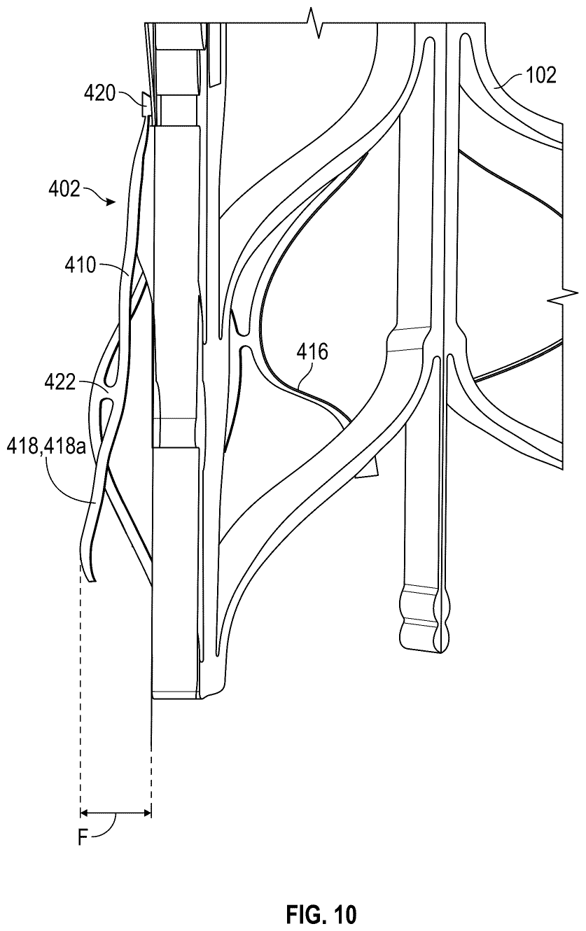

The free portions of the skirt frame 402 that are not fixed to the valve frame 102 (for example, including the free lower apices 418b and the free upper apices 420b, the intermediate portions of the struts 410 between the apices 418, 420, etc.) can be configured to flex or bulge outwardly in a radial direction, away from the valve frame 102. In particular, when the valve frame 102 is radially expanded, the free portions of the skirt frame 402 can be radially separated from the valve frame 102 by a flex distance F (FIG. 10). The flex distance F can be dependent on the extent to which the valve frame 102 is radially expanded. The flex distance F can also be impacted by the shape, pattern, orientation (for example, twisted, flat, etc.) of the struts of the skirt frame 402 as well as the locations where the skirt frame 402 is attached to the valve frame 102. As such, the skirt frame 402 can be configured to expand further away from the valve frame 102 in a controlled manner, such that the desired profile, size, and locations of the bulging portions of the skirt frame 402 can be predetermined.

In some examples, as depicted, junctions 422 between the cells 416 can be configured to twist outwardly in a radial direction when the skirt frame 402 is in a radially expanded state (see FIG. 10). Specifically, when twisted, the width of the junctions 422 can be oriented radially outward when the skirt frame 402 is in the radially expanded state. As such, the width of the junctions 422 can add to the amount that the skirt frame 402 bulges or flexes radially outward from the valve frame 102, thus enlarging the radial profile of the skirt frame 402.

As described above, the skirt 404 can be coupled to the skirt frame 402. As such, when the skirt frame 402 radially bulges or flexes away from the valve frame 102, the skirt 404 is also bulged or flexed outwardly away from the valve frame 102 in the radial direction. The radial bulging or flexing of the skirt 404 can help improve the sealing of the prosthetic valve 100 against the tissue of the native valve annulus and help reduce paravalvular leakage past the prosthetic valve 100.

In some examples, the skirt frame 402 can be relatively thin (for example, having a strut thickness of 0.08 mm or less, preferably 0.02-0.05 mm, etc.), such that the skirt frame 402 does not significantly add to the overall outer diameter of the prosthetic valve 100 when the valve frame 102 is in a crimped or radially compressed state (FIG. 9). In this way, the skirt frame 402 advantageously preserves the crimped profile of the valve frame 102, while also bulging radially outward when the valve frame 102 is in the radially expanded state to improve sealing against the native tissue upon implantation and reduce paravalvular leakage.

Each of the valve frame 102 and the skirt frame 402 can be a unitary and/or fastener-free frame that can be constructed from a single piece of material (for example, Nitinol, stainless steel or a cobalt-chromium alloy), such as in the form of a tube. The plurality of struts and/or cells can be formed by removing portions (for example, via laser cutting) of the single piece of material. In some examples, the skirt frame 402 may be laser-cut from a thin tube of material. For example, the skirt frame 402 can be cut from a tube having a wall thickness of 0.08 mm and undergo an electropolish process which can further thin the skirt frame 402 to a thickness in the range of about 0.02-0.05 mm.

In some examples, the skirt frame 402 may be cut from a relatively narrow tube having a diameter the same or close to the radially compressed state of the skirt frame (for example, the tube can have a diameter of 6-7 mm, etc.), such that the skirt frame 402 is in a normally-radially compressed state. For example, when the prosthetic valve 100 is crimped or radially compressed, the skirt frame 402 naturally assumes its free state, being tightly compressed over the valve frame 102. In examples where the skirt frame 402 is formed from a shape-memory material (for example, Nitinol), the skirt frame can be shape set in the radially compressed state (that is, biased to the radially compressed state) and the free state of the skirt frame 402 is also referred to as a shape-memory state or a shape set state. Additionally, radially expanding the valve frame 102 results in deformation of the skirt frame 402 from the shape-memory state to a deformed state (for example, a radially expanded state).

In other examples, the skirt frame 402 can be formed from a shape-memory material (for example, Nitinol) and can be shape set in the radially expanded state (that is, biased to the radially expanded state). In such examples, the skirt frame 402 can be retained in the radially compressed state during delivery by a restraining force (such as by a delivery capsule extending over the prosthetic valve). Releasing the restraining force (such as by advancing the prosthetic valve from the delivery capsule) allows the skirt frame 402 to fully self-expand to the radially expanded state, or to self-expand to a partially radially expanded state. As such, in some examples, the flex distance F (FIG. 10) may not be impacted by the degree of expansion of the valve frame 102. When partially expanded, radially expanding the valve frame 102 can cause the skirt frame 402 to further expand from the partially expanded state to the fully expanded state.

The strains experienced by the skirt frame 402 are configured to be in the elastic range. For example, the skirt frame 402 can experience a 0.3% strain in the crimped state (FIG. 9). The skirt frame 402 can experience strains of 5.5% during valve expansion to 27 mm and strains of 6.2% when expanded to 30 mm (FIGS. 6-8). Strains in the elastic range allow the skirt frame 402 to flexibly revert back to the compressed state when the valve frame 102 is crimped or re-compressed.

In some examples, the valve frame 102 and/or the skirt frame 402 can be formed from a plastically-expandable material, such as stainless steel or a cobalt-chromium alloy. When the frames are formed from a plastically-expandable material, the prosthetic valve 100 can be placed in a radially compressed state along the distal end portion of a delivery apparatus for insertion into a patient's body (FIG. 9). When at the desired implantation site, the valve frame 102 (and therefore the prosthetic valve 100) can be radially expanded from the radially compressed state to a radially expanded state via mechanical actuation (for example, rotation of actuators 126 to produce expansion of the valve frame 102) or inflation of a balloon. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In other examples, the valve frame 102 and/or the skirt frame 402 can be formed from a shape-memory material (for example, Nitinol). When formed from a shape-memory (or self-expandable) material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule and can self-expand to a radially expanded state. In some examples, the valve frame 102 (and therefore the prosthetic valve 100) can partially self-expand from the radially compressed state to a partially radially expanded state. The valve frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

In some examples, the valve frame 102 can be formed from a plastically-expandable material and the skirt frame 402 can be formed from a shape-memory material. In other examples, the skirt frame 402 can be formed from a plastically-expandable material and the valve frame 102 can be formed from a shape-memory material.

While the skirt frame 402 is coupled to a mechanically expandable frame (for example, valve frame 102) in FIGS. 6-10, it should be appreciated that the skirt frame 402 can be coupled to other valve frames, including those that are expandable in other manners (for example, self-expandable, balloon-expandable, etc.). For example, the skirt frame 402 can be coupled to other radially expandable valve frames including any of those described herein (for example, valve frame 502, which desirably is configured to be expanded by inflation of a balloon, etc.).

FIG. 11 shows a prosthetic heart valve 500 (prosthetic valve), according to another example. The prosthetic heart valve 500 can include a stent or frame 502, a valvular structure 504, and a perivalvular outer sealing member or outer skirt 506. The prosthetic heart valve 500 (and the frame 502) can have an inflow end 508 and an outflow end 510. The valvular structure 504 can be disposed on an interior of the frame 502 while the outer skirt 506 is disposed around an outer surface of the frame 502.

The valvular structure 504 can comprise a plurality of leaflets 512 (for example, three leaflets, as shown in FIG. 11), collectively forming a leaflet structure, which can be arranged to collapse in a tricuspid arrangement. The leaflets 512 can be secured to one another at their adjacent sides (for example, commissure tabs) to form commissures 514 of the valvular structure 504. For example, each leaflet 512 can comprise opposing commissure tabs disposed on opposite sides of the leaflet 512 and a cusp edge portion extending between the opposing commissure tabs. The cusp edge portion of the leaflets 512 can have an undulating, curved scalloped shape, and can be secured directly to the frame 502 (for example, by sutures). However, in alternate examples, the cusp edge portion of the leaflets 512 can be secured to an inner skirt which is then secured to the frame 502. In some examples, the leaflets 512 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

In some examples, the outer skirt 506 can be an annular skirt. In some examples, the outer skirt 506 can comprise one or more skirt portions that are connected together and/or individually connected to the frame 502. The outer skirt 506 can comprise a fabric or polymeric material, such as ePTFE, PTFE, PET, TPU, UHMWPE, PEEK, PE, etc. In some examples, instead of having a relatively straight upper edge portion, as shown in FIG. 11, the outer skirt 506 can have an undulating upper edge portion that extends along and is secured to the angled struts 534. Examples of such outer skirts, as well as various other outer skirts, that can be used with the frame 502 can be found in the provisional patent application under Edwards attorney docket No. 12131US01, which is incorporated by reference herein.

The frame 502 can be radially compressible and expandable between a radially compressed state and a radially expanded state (the expanded state is shown in FIG. 11). The frame 502 is shown alone in FIG. 12 and a portion of the frame 502 in a straightened (non-annular) state is shown in FIG. 13.

The frame 502 can be made of any of various suitable plastically-expandable materials (for example, stainless steel, etc.) or self-expanding materials (for example, nickel titanium alloy (NiTi), such as nitinol). When constructed of a plastically-expandable material, the frame 502 (and thus the valve 500) can be crimped to a radially compressed state on a delivery catheter and then expanded inside a patient by an inflatable balloon or equivalent expansion mechanism. When constructed of a self-expandable material, the frame 502 (and thus the valve 500) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frame 502 include, without limitation, stainless steel, a nickel-based alloy (for example, a cobalt-chromium or a nickel-cobalt-chromium alloy), polymers, or combinations thereof. In particular examples, frame 502 can be made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIGS. 12 and 13, the frame 502 can comprise a plurality of interconnected struts 516 which form multiple rows of open cells 518 between the outflow end 510 and the inflow end 508 of the frame 502. In some examples, as shown in FIGS. 12 and 13, the frame 502 can comprise three rows of cells 518 with a first (upper in the orientation shown in FIGS. 12 and 13) row of cells 520 disposed at the outflow end 510. The first row of cells 520 comprises cells 518 that are elongated in an axial direction (relative to a central longitudinal axis 522 of the frame 502), as compared to cells 518 in the remaining rows of cells. For example, the cells 518 of the first row of cells 520 can have a longer axial length 524 (FIG. 13) than cells 518 in the remaining rows of cells, which can include a second row of cells 526 and a third row of cells 528, the third row of cells 528 disposed at the inflow end 508 and the second row of cells 526 disposed between the first row of cells 520 and the third row of cells 528.

In some examples, as shown in FIG. 12, each row of cells comprises nine cells 518. Thus, in such examples, the frame 502 can be referred to as a nine-cell frame.

In alternate examples, the frame 502 can comprise more than three rows of cells (for example, four or five) and/or more or less than nine cells per row. In some examples, the cells 518 in the first row of cells 520 may not be elongated compared to cells 518 in the remaining rows of cells of the frame 502 (the second row of cells 526 and the third row of cells 528).

The interconnected struts 516 can include a plurality of angled struts 530, 532, 534, and 536 arranged in a plurality of rows of circumferentially extending rows of angled struts, with the rows being arrayed along the length of the frame 502 between the outflow end 510 and the inflow end 508. For example, the frame 502 can comprise a first row of angled struts 530 arranged end-to-end and extending circumferentially at the inflow end 508 of the frame; a second row of circumferentially extending, angled struts 532; a third row of circumferentially extending, angled struts 534; and a fourth row of circumferentially extending, angled struts 536 at the outflow end 510 of the frame 502. The fourth row of angled struts 536 can be connected to the third row of angled struts 534 by a plurality of axially extending window struts 538 (or window strut portions) and a plurality of axial (for example, axially extending) struts 540. The axially extending window struts 538 (which can also be referred to as axial struts that include a commissure window) define commissure windows (for example, open windows) 542 that are spaced apart from one another around the frame 502, in a circumferential direction, and which are adapted to receive a pair of commissure tabs of a pair of adjacent leaflets 512 arranged into a commissure (for example, commissure 514 shown in FIG. 11). In some examples, the commissure windows 542 and/or the axially extending window struts 538 defining the commissure windows 542 can be referred to herein as commissure features or commissure supports, each commissure feature or support configured to receive and/or be secured to a pair of commissure tabs of a pair of adjacent leaflets.

One or more (for example, two, as shown in FIGS. 12 and 13) axial struts 540 can be positioned between, in the circumferential direction, two commissure windows 542 formed by the window struts 538. Since the frame 502 can include fewer cells per row (for example, nine) and fewer axial struts 540 between each commissure window 542, as compared to some more traditional prosthetic heart valves, each cell 518 can have an increased width (in the circumferential direction), thereby providing a larger opening for blood flow and/or coronary access.

Each axial strut 540 and each window strut 538 extends from a location defined by the convergence of the lower ends (for example, ends arranged inward of and farthest away from the outflow end 510) of two angled struts 536 (which can also be referred to as an upper strut junction or upper elongated strut junction) to another location defined by the convergence of the upper ends (for example, ends arranged closer to the outflow end 510) of two angled struts 534 (which can also be referred to as a lower strut junction or lower elongate strut junction). Each axial strut 540 and each window strut 538 forms an axial side of two adjacent cells of the first row of cells 520.

In some examples, as shown in FIG. 13, each axial strut 540 can have a width 544 (FIG. 13) that is larger than a width of the angled struts 530, 532, 534, and 536. As used herein, a “width” of a strut is measured between opposing locations on opposing surfaces of a strut that extend between the radially facing inner and outer surfaces of the strut (relative to the central longitudinal axis 522 of the frame 502). A “thickness” of a strut is measured between opposing locations on the radially facing inner and outer surfaces of a strut and is perpendicular to the width of the strut. In some examples, the width 544 of the axial struts 540 is 50-200%, 75-150%, or at least 100% larger than (for example, double) the width of the angled struts of the frame 502.

By providing the axial struts 540 with the width 544 that is greater than the width of other, angled struts of the frame 502, a larger contact area is provided for when the leaflets 512 contact the wider axial struts 540 during systole, thereby distributing the stress and reducing the extent to which the leaflets 512 may fold over the axial struts 540, radially outward through the cells 518. As a result, a long-term durability of the leaflets 512 can be increased.

Since the cells 518 of the frame 502 can have a relatively large width compared to alternate prosthetic valves that have more than nine cells per row (as introduced above), the wider axial struts 540 can be more easily incorporated into the frame 502, without sacrificing open space for blood flow and/or coronary access.

Commissure tabs 515 of adjacent leaflets 512 can be secured together to form commissures 514 (FIG. 11). Each commissure 514 of the prosthetic heart valve 500 comprises two commissure tabs 515 paired together, one from each of two adjacent leaflets 512, and extending through a commissure window 542 of the frame 502. Each commissure 514 can be secured to the window struts 538 forming the commissure window 542.

The cusp edge portion (for example, scallop edge) of each leaflet 512 can be secured to the frame 502 via one or more fasteners (for example, sutures). In some examples, the cusp edge portion of each leaflet 512 can be secured directly to the struts of the frame 502 (for example, angled struts 530, 532, and 534). For example, the cusp edge portions of the leaflets 512 can be sutured to the angled struts 530, 532, and 534 that generally follow the contour of the cusp edge portions of the leaflets 512.

In some examples, the cusp edge portion of the leaflets 512 can be secured to an inner skirt and the inner skirt can then be secured directly to the frame 502.

Various methods for securing the leaflets 512 to a frame, such as the frame 502, are disclosed in U.S. provisional patent applications 63/278,922, filed Nov. 12, 2021, and 63/300,302, filed Jan. 18, 2022, both of which are incorporated by reference herein.

As shown in FIGS. 12 and 13, in some examples, one or more of or each of the axial struts 540 can comprise an inflow end portion 546 (for example, an end portion that is closest to the inflow end 508) and an outflow end portion 548 that are widened relative to a middle portion 550 of the axial strut 540 (which can be defined by the width 544). In some examples, the inflow end portion 546 of the axial strut 540 can comprise an aperture 547. The apertures 547 can be configured to receive fasteners (for example, sutures) for attaching soft components of the prosthetic heart valve 500 to the frame 502. For example, the outer skirt 506 can be positioned around the outer surface of the frame 502 and an upper or outflow edge portion of the outer skirt 506 can be secured to the apertures 547 by fasteners 549 (for example, sutures), as shown in FIG. 11.

The frame 502 can further comprise a plurality of apex regions 552 formed at the inflow end 508 and the outflow end 510, each apex region 552 extending and forming a junction between two angled struts 530 at the inflow end 508 or two angled struts 536 at the outflow end 510. As such, the apex regions 552 are spaced apart from one another, in a circumferential direction at the inflow end 508 and the outflow end 510.

Each apex region 552 can comprise an apex 554 (the highest or most outward extending, in an axial direction, point) and two thinned (or narrowed) strut portions 556, one thinned strut portion 556 extending from either side of the apex 554 to a corresponding, wider, angled strut 536 (at the outflow end 510) or angled strut 530 (at the inflow end 508) (FIG. 13). In this way, each of the apex regions 552 at the outflow end 510 can form a narrowed transition region between and relative to the two angled struts 536 extending from the corresponding apex region 552 and each of the apex regions 552 at the inflow end 508 can form a narrowed transition region between and relative to the two angled struts 530 extending from the corresponding apex region 552.

The thinned strut portions 556 of the apex regions 552 can have a width 558 that is smaller than a width 560 of the angled struts 530 or 536 (FIG. 13). In some examples, the width 558 can be a uniform width (for example, along an entire length of the strut portion 556). In some examples, the width 558 of the thinned strut portions 556 can be from about 0.06-0.15 mm smaller than the width 560 of the angled struts 530 and/or 536.

The thinned strut portions 556 of the apex regions 552 can have a first length 562 (FIG. 13). In some examples, the first length 562 is in a range of 0.8-1.4 mm, 0.9-1.2 mm, 0.95-1.05 mm, or about 1.0 mm (for example, ±0.03 mm). In alternate examples, the first length 562 is in a range of 0.3-0.7 mm, 0.4-0.6 mm, 0.45-0.55 mm, or about 0.5 mm (for example, ±0.03 mm).

Thus, each outflow apex region 552 can include two thinned strut portions 556 having the first length 562, each extending from the apex 554, outward relative to a central longitudinal axis 564 of the cells 518. Thus, a total length of the apex region 552 can be two times the first length 562.

Each apex region 552 and two corresponding angled struts 536 at the outflow end 510 can form an outflow strut 566 and each apex region 552 and two corresponding angled struts 530 at the inflow end 508 can form an inflow strut 568.

Each outflow strut 566 and inflow strut 568 can have a length that includes an apex region 552 and the two angled struts 536 or 530 (or strut portions), respectively, on either side of the apex region 552. One half the total length of each outflow strut 566 and inflow strut 568 is shown in FIG. 13 as length 570, which extends from an end of one angled strut 536 or 530 to the central longitudinal axis 564. Thus, the length of each outflow strut 566 and inflow strut 568 is two times length 570. In some examples, the length 570 for half of each inflow strut 568 can be different than the length 570 for half of each outflow strut 566.

In some examples, the length of each thinned strut portion 556 can be at least 25% of the length 570 of the corresponding half outflow strut 566 or inflow strut 568. Said another way, the length of each apex region 552 (a total length being two times the first length 562) can be at least 25% of the total length (two times length 570) of the outflow strut 566 or inflow strut 568. In some examples, the length of each apex region 552 can be more than 25% of the total length of the corresponding outflow strut 566 or inflow strut 568, such as 25-35%.

In some examples, each apex region 552 can comprise a curved, axially facing outer surface 572 and an arcuate or curved, axially facing inner depression 574 which forms the thinned strut portions 556. For example, the curved inner depression 574 can depress toward the curved outer surface 572 from an inner surface of the angled strut portions 556, thereby forming the smaller width thinned strut portions 556. Thus, the curved inner depressions 574 can be formed on a cell side of the apex region 552 (for example, as opposed to the outside of the apex region 552).

In some examples, the curved outer surface 572 of each apex region 552 can form a single, continuous curve from one angled strut portion 556 on a first side of the apex region 552 to another angled strut portion 556 on an opposite, second side of the apex region 552.

Each apex region 552 can have a radius of curvature 576, along the curved outer surface 572 (for example, along an entirety or an entire length of the curved outer surface 572) (FIG. 13). In some examples, the radius of curvature 576 at the apex 554 and/or along the entire curved outer surface 572 of the apex region 552 can be greater than 1 mm. In some examples, the radius of curvature 576 can be in a range of 1-20 mm, 3-16 mm, or 8-14 mm. In some examples, the radius of curvature 576 can be greater than 10 mm. The radius of curvature 576 can be dependent on (and thus change due to changes in) the width 558 (for example, the amount of reduction in width from the angled struts 530 or 536) and the first length 562 of the thinned strut portions 556.

Further, a height (an axial height) 578 of the apex regions 552, which can be defined in the axial direction from an outer surface of the two angled struts 530 or 536 to the curved outer surface 572 of the apex region 552 at the apex 554, can be the width 558 of the thinned strut portions 556 (FIG. 13). In this way, the height 578 of the apex regions 552 can be relatively small and not add much to the overall axial height of the radially expanded frame 502. Thus, the leaflets 512 secured to the frame 502 (FIG. 11) can be disposed close to the inflow end 508, thereby leaving a larger open space at the outflow end 510 of the frame 502 that is not blocked by the leaflets 512.

In some examples, each of the apex region 552 can form an angle 580 between the two angled struts 530 or 536 extending from either side of the corresponding apex region 552 (FIG. 13). In some examples, the angle 580 can be in a range of 120 (not inclusive) to 140 degrees (for example, such that the angle 580 is greater than 120 degrees and less than or equal to 140 degrees).

Additional details and examples of frames for prosthetic heart valves that include apex regions can be found in U.S. Provisional Patent Application Nos. 63/178,416, filed Apr. 22, 2021, 63/194,830, filed May 28, 2021, and 63/279,096, filed Nov. 13, 2021, all of which are incorporated by reference herein.

FIG. 14 shows a delivery apparatus 600, according to an example, that can be used to implant an expandable prosthetic heart valve (for example, the prosthetic heart valve 500 of FIG. 11 and/or any of the other prosthetic heart valves described herein). In some examples, the delivery apparatus 600 is specifically adapted for use in introducing a prosthetic valve into a heart.

The delivery apparatus 600 in the illustrated example of FIG. 14 is a balloon catheter comprising a handle 602 and a steerable, outer shaft 604 extending distally from the handle 602. The delivery apparatus 600 can further comprise an intermediate shaft 606 (which also may be referred to as a balloon shaft) that extends proximally from the handle 602 and distally from the handle 602, the portion extending distally from the handle 602 also extending coaxially through the outer shaft 604. Additionally, the delivery apparatus 600 can further comprise an inner shaft 608 extending distally from the handle 602 coaxially through the intermediate shaft 606 and the outer shaft 604 and proximally from the handle 602 coaxially through the intermediate shaft 606.

The outer shaft 604 and the intermediate shaft 606 can be configured to translate (for example, move) longitudinally, along a central longitudinal axis 620 of the delivery apparatus 600, relative to one another to facilitate delivery and positioning of a prosthetic valve at an implantation site in a patient's body.

The intermediate shaft 606 can include a proximal end portion 610 that extends proximally from a proximal end of the handle 602, to an adaptor 612. A rotatable knob 614 can be mounted on the proximal end portion 610 and can be configured to rotate the intermediate shaft 606 around the central longitudinal axis 620 and relative to the outer shaft 604.

The adaptor 612 can include a first port 638 configured to receive a guidewire therethrough and a second port 640 configured to receive fluid (for example, inflation fluid) from a fluid source. The second port 640 can be fluidly coupled to an inner lumen of the intermediate shaft 606.

The intermediate shaft 606 can further include a distal end portion that extends distally beyond a distal end of the outer shaft 604 when a distal end of the outer shaft 604 is positioned away from an inflatable balloon 618 of the delivery apparatus 600. A distal end portion of the inner shaft 608 can extend distally beyond the distal end portion of the intermediate shaft 606.

The balloon 618 can be coupled to the distal end portion of the intermediate shaft 606.

In some examples, a distal end of the balloon 618 can be coupled to a distal end of the delivery apparatus 600, such as to a nose cone 622 (as shown in FIG. 14), or to an alternate component at the distal end of the delivery apparatus 600 (for example, a distal shoulder). An intermediate portion of the balloon 618 can overlay a valve mounting portion 624 of a distal end portion of the delivery apparatus 600 and a distal end portion of the balloon 618 can overly a distal shoulder 626 of the delivery apparatus 600. The valve mounting portion 624 and the intermediate portion of the balloon 618 can be configured to receive a prosthetic heart valve in a radially compressed state. For example, as shown schematically in FIG. 14, a prosthetic heart valve 650 (which can be one of the prosthetic valves described herein) can be mounted around the balloon 618, at the valve mounting portion 624 of the delivery apparatus 600.

The balloon shoulder assembly, including the distal shoulder 626, is configured to maintain the prosthetic heart valve 650 (or other medical device) at a fixed position on the balloon 618 during delivery through the patient's vasculature.

The outer shaft 604 can include a distal tip portion 628 mounted on its distal end. The outer shaft 604 and the intermediate shaft 606 can be translated axially relative to one another to position the distal tip portion 628 adjacent to a proximal end of the valve mounting portion 624, when the prosthetic valve 650 is mounted in the radially compressed state on the valve mounting portion 624 (as shown in FIG. 14) and during delivery of the prosthetic valve to the target implantation site. As such, the distal tip portion 628 can be configured to resist movement of the prosthetic valve 650 relative to the balloon 618 proximally, in the axial direction, relative to the balloon 618, when the distal tip portion 628 is arranged adjacent to a proximal side of the valve mounting portion 624.

An annular space can be defined between an outer surface of the inner shaft 608 and an inner surface of the intermediate shaft 606 and can be configured to receive fluid from a fluid source via the second port 640 of the adaptor 612. The annular space can be fluidly coupled to a fluid passageway formed between the outer surface of the distal end portion of the inner shaft 608 and an inner surface of the balloon 618. As such, fluid from the fluid source can flow to the fluid passageway from the annular space to inflate the balloon 618 and radially expand and deploy the prosthetic valve 650.

An inner lumen of the inner shaft can be configured to receive a guidewire therethrough, for navigating the distal end portion of the delivery apparatus 600 to the target implantation site.

The handle 602 can include a steering mechanism configured to adjust the curvature of the distal end portion of the delivery apparatus 600. In the illustrated example, for example, the handle 602 includes an adjustment member, such as the illustrated rotatable knob 660, which in turn is operatively coupled to the proximal end portion of a pull wire. The pull wire can extend distally from the handle 602 through the outer shaft 604 and has a distal end portion affixed to the outer shaft 604 at or near the distal end of the outer shaft 604. Rotating the knob 660 can increase or decrease the tension in the pull wire, thereby adjusting the curvature of the distal end portion of the delivery apparatus 600. Further details on steering or flex mechanisms for the delivery apparatus can be found in U.S. Pat. No. 9,339,384, which is incorporated by reference herein.

The handle 602 can further include an adjustment mechanism 661 including an adjustment member, such as the illustrated rotatable knob 662, and an associated locking mechanism including another adjustment member, configured as a rotatable knob 678. The adjustment mechanism 661 is configured to adjust the axial position of the intermediate shaft 606 relative to the outer shaft 604 (for example, for fine positioning at the implantation site). Further details on the delivery apparatus 600 can be found in PCT Application No. PCT/US2021/047056, which is incorporated by reference herein.

In some examples, the outer skirt 506 of the prosthetic heart valve 500 can be replaced with a skirt assembly (for example, any of the skirt assemblies described herein). For example, as shown in FIG. 15, the prosthetic heart valve 500 may include a skirt assembly 700 (also referred to as a “sealing assembly 700”) mounted around the frame 502 (also referred to as a “valve frame 502”). The skirt assembly 700 can function as a scaling mechanism for the prosthetic valve 500 by sealing against the tissue of the native valve annulus and helping to reduce paravalvular leakage past the prosthetic valve 500. For example, as shown in FIG. 15, the skirt assembly 700 may include a skirt frame 702 (also referred to as a sealing frame) and a skirt 704 (also referred to as a sealing member or scaling layer) mounted around an outer surface of the valve frame 502. FIG. 16 shows the skirt frame 702 by itself.

As shown in FIG. 15, the skirt assembly 700 may include a skirt 704 that is coupled to the skirt frame 702. For example, the skirt 704 can be coupled to the skirt frame 702 in any manner described above in connection with coupling the skirt 404 to the skirt frame 402. Similar to the skirt 404, the skirt 704 can function as a sealing member for the prosthetic valve 500 by sealing against the tissue of the native valve annulus and helping to reduce paravalvular leakage past the prosthetic valve 500. As will be described in more detail below, the skirt frame 702 can be configured to radially bulge or flex away from the valve frame 502 to improve scaling of the prosthetic valve 500 against the tissue of the native valve annulus and help reduce paravalvular leakage.

The skirt frame 702 can be coupled (for example, fixedly mounted) to an outer surface of the valve frame 502. For example, the skirt frame 702 can be coupled to the valve frame 502 in any manner described above in connection with coupling the skirt frame 402 to the valve frame 102.

The skirt frame 702 can be made of any of various suitable plastically expandable materials (for example, stainless steel, etc.) or self-expanding materials (for example, Nitinol), similar to skirt frame 402 as described above.

The skirt frame 702 can be mounted to any number of struts of the valve frame 502. In some examples, as depicted, portions of the skirt frame 702 can be fixed to each of the inflow struts 568 as well as to the axial struts 538, 540. As shown, the skirt frame 702 is positioned towards the inflow end 508 of the valve frame 502. In particular, a first (or inflow) end 706 of the skirt frame 702 is coupled to the inflow struts 568 (for example, to the apices 554 at the inflow end 508) and a second (or outflow) end 708 of the skirt frame 702 is coupled to the inflow end portions 546 of the axial struts 540 and inflow end portions of the axial struts 538 (for example, between inflow and outflow ends 508, 510 of the valve frame 502). For example, as depicted, the second end 708 of the skirt frame 702 is coupled to the valve frame 502 at a location where struts 534 of the cells 518 (FIG. 13) connect to the axial struts 538, 540 (for example, positioned closer to the inflow end 508 than the commissure windows 542, etc.). It should be noted that the skirt frame 702 can be mounted at any locations along the length of the valve frame 502 and/or can have a length (measured from the inflow end 706 to the outflow end 708) that is less than or equal to the length of the valve frame, as described above for the skirt frame 402.

The skirt frame 702 can include a plurality of circumferentially extending link members or struts 710. As illustrated in FIGS. 15-16, the struts 710 can include single row of interconnected struts in a zig-zag pattern that extends circumferentially around the skirt frame 702. Specifically, each strut 710 extends between the first and second ends 706, 708 of the skirt frame 702. The struts 710 can define a plurality of inflow apices 712 at the first end 706 of the skirt frame 702 and a plurality of outflow apices 714 at the second end 708 of the skirt frame 702. In some examples, as depicted, each of the inflow and outflow apices 712, 714 are coupled to the valve frame 502 and can be referred to as “fixed” inflow and outflow apices 712, 714. In other examples, only some of the inflow and outflow apices 712, 714 are coupled to the valve frame 502 (for example, at least one fixed inflow apex 712 and at least one fixed outflow apex 714, etc.). The struts 710 can include intermediate (or free) portions 716 that are not fixed or attached to the valve frame 502. In some examples, as depicted, the fixed inflow and outflow apices 712, 714 have T-bars for connecting the apices to the valve frame 502. For example, a suture can be wrapped around T-bars or similar connection features of the fixed inflow and outflow apices 712, 714 to connect the apices 712, 714 to the posts of the valve frame 502.

The inflow apices 712 can be circumferentially offset from the outflow apices 714. In this manner, the location(s) where the first end 706 of the skirt frame 702 is coupled to the valve frame 502 is staggered or offset circumferentially from the location(s) where the second end 708 of the skirt frame 702 is coupled to the valve frame 502.

Similar to the skirt frame 402, the skirt frame 702 can be configured to radially bulge or flex away from the valve frame 502 to improve sealing of the prosthetic valve 500 against the tissue of the native valve annulus and help reduce paravalvular leakage. In particular, intermediate (or free) portions 716 of the struts 710 (that is, the portions of the struts 710 that are not fixed or attached to the valve frame 502) radially bulge or flex away from the valve frame 502 when the prosthetic heart valve 500 is radially expanded. Specifically, during radial expansion, the valve frame 502 and the skirt frame 702 foreshorten along their respective axial lengths which results in the skirt frame 702 (for example, the free portions 716, etc.) radially bulging or flexing away from the valve frame 502 by a flex amount, adding to the overall radial profile of the prosthetic heart valve 500 when radially expanded.

In some examples, as shown in FIG. 16, each strut 710 can have a width 718 (FIG. 16) that is larger than a thickness 720 of the strut 710. As used herein, a “width” of a strut is measured between opposing locations on opposing surfaces of a strut that extend between the radially facing inner and outer surfaces of the strut (relative to a central longitudinal axis of the skirt frame 702) before any shape setting or other deformation of the struts to place the struts in a twisting configuration (described below). Thus, before any twisting of the struts, the width is generally perpendicular to a line that extends radially from the central axis of the skirt frame to the skirt frame. A “thickness” of a strut is measured between opposing locations on the radially facing inner and outer surfaces of a strut before any shape setting or other deformation of the struts to place the struts in a twisting configuration and is perpendicular to the width of the strut. In some examples, the width 718 of each strut 710 can be significantly greater than the thickness 720 of each strut 710 (for example, at least three times greater, etc.).

In some examples, as depicted in FIGS. 15-16, the free portion 716 of each strut 710 can be twisted between the first and second ends 706, 708 of the skirt frame 702 (for example, each strut 710 is twisted between an inflow apex 712 and an outflow apex 714). For example, a strut 710 can be twisted 90 degrees in a first direction starting at one apex 712 to an intermediate location of the strut and then twisted 90 degrees in a second direction from the intermediate location to an apex 714. When twisted, the width 718 of the strut 710 can be oriented in a radial direction (for example, facing in the radial or circumferential direction) when the skirt frame 702 is in a radially expanded state. As such, the width 718 of the strut 710 can add to the amount that the skirt frame 702 bulges or flexes radially outward from the valve frame 502, thus enlarging the radial profile of the skirt frame 702. In some examples, as depicted, the struts 710 can include a partial twist between the inflow and outflow apices 712, 714. In these examples, the skirt frame 702 can be formed from a single piece of material, as described above. In other examples, such as when the struts 710 are separately formed and then joined to each other to form the skirt frame 702, each strut 710 can include one or more full twists between the inflow and outflow apices 712, 714.

In some examples, the skirt frame 702 can be relatively thin, such that the skirt assembly 700 does not significantly add to the overall outer diameter of the valve frame 502 when the prosthetic heart valve 500 is in a crimped or radially compressed state (such as for delivery). For example, the thickness 720 of each strut 710 can be, for example, 0.08 mm or less and preferably 0.02 mm to 0.05 mm. In this way, the skirt frame 702 advantageously preserves the crimped profile of the valve frame 502, while also bulging radially outward when the valve frame 502 is in the radially expanded state to improve scaling against the native tissue upon implantation and reduce paravalvular leakage.

As described above, the skirt 704 can be coupled to the skirt frame 702. Accordingly, when the skirt frame 702 radially bulges or flexes away from the valve frame 502, the skirt 704 is also bulged or flexed outwardly in the radial direction. The radial bulging or flexing of the skirt 704 can help improve the sealing of the prosthetic heart valve 500 against the tissue of the native valve annulus and help reduce paravalvular leakage past the prosthetic valve.

FIG. 17 illustrates another example of a skirt assembly 800 that is coupled to a prosthetic heart valve (for example, prosthetic heart valve 100 of FIG. 1A, prosthetic heart valve 500 of FIG. 11 (for example, in lieu of outer skirt 506), and/or any of prosthetic heart valve described herein). In particular, as depicted, the skirt assembly 800 is coupled to valve frame 502 of the prosthetic heart valve 500. The skirt assembly 800 can function as a scaling mechanism for the prosthetic valve 500 by sealing against the tissue of the native valve annulus and helping to reduce paravalvular leakage past the prosthetic valve 500. For example, as shown in FIG. 17, the skirt assembly 800 may include a skirt frame 802 (also referred to as a sealing frame) and a skirt 804 (also referred to as a sealing member) mounted around an outer surface of the valve frame 502.

As shown in FIG. 17, the skirt assembly 800 may include a skirt 804 that is coupled to the skirt frame 802. For example, the skirt 804 can be coupled to the skirt frame 802 in any manner described above in connection with coupling the skirt 404 to the skirt frame 402. Similar to the skirt 404, the skirt 804 can function as a sealing member for the prosthetic valve 500 by sealing against the tissue of the native valve annulus and helping to reduce paravalvular leakage past the prosthetic valve 500. As will be described in more detail below, the skirt frame 802 can be configured to radially bulge or flex away from the valve frame 502 to improve sealing of the prosthetic valve 500 against the tissue of the native valve annulus and help reduce paravalvular leakage.

The skirt frame 802 can be coupled (for example, fixedly mounted) to an outer surface of the valve frame 502. For example, the skirt frame 802 can be coupled to the valve frame 502 in manners similar to those described above.

The skirt frame 802 can be made of any of various suitable plastically expandable materials (for example, stainless steel, etc.) or self-expanding materials (for example, Nitinol), similar to skirt frame 402 as described above.

The skirt frame 802 can be mounted to any number of struts of the valve frame 502. In some examples, portions of the skirt frame 802 can be fixed to each of the inflow struts 568 (FIG. 13) as well as to the axial struts 538, 540. As shown, the skirt frame 802 is positioned towards the inflow end 508 of the valve frame 502. In particular, a first (or inflow) end 806 of the skirt frame 802 is coupled to the inflow struts 568 (for example, to the apices 554 at the inflow end 508) and a second (or outflow) end 808 of the skirt frame 802 is coupled to the inflow end portions 546 of the axial struts 540 and inflow end portions of the axial struts 538 (for example, between inflow and outflow ends 508, 510 of the valve frame 502). For example, as depicted, the second end 808 of the skirt frame 802 is coupled to the valve frame 502 at a location where struts 534 of the cells 518 connect to the axial struts 538, the axial struts 540 (for example, positioned closer to the inflow end 508 than the commissure windows 542, etc.).

The skirt frame 802 can include a plurality of circumferentially extending, interconnected link members or struts. As illustrated in FIG. 17, the struts can include first row of struts 810 in a zig-zag pattern that extends circumferentially around the skirt frame 802. Specifically, each strut 810 extends between the first and second ends 806, 808 of the skirt frame 802. The struts 810 can define a plurality of inflow apices 812 at the first end 806 of the skirt frame 802 and a plurality of outflow apices 814 at the second end 808 of the skirt frame 802. In some examples, as depicted, each of the inflow and outflow apices 812, 814 are coupled to the valve frame 502 and can be referred to as “fixed” inflow and outflow apices 812, 814. In other examples, only some of the inflow and outflow apices 812, 814 are coupled to the valve frame 502 (for example, at least one fixed inflow apex 812 and at least one fixed outflow apex 814, etc.). The struts 810 can include intermediate (or free) portions 816 that are not fixed or attached to the valve frame 502.

The inflow apices 812 can be circumferentially offset from the outflow apices 814. In this manner, the location(s) where the first end 806 of the skirt frame 802 is coupled to the valve frame 502 are staggered or offset circumferentially from the location(s) where the second end 808 of the skirt frame 802 is coupled to the valve frame 502.

The skirt frame 802 can also include a second row of struts 811 at or near the second end 808 of the skirt frame 802. The struts 811 can extend from the intermediate portions 816 of the struts 810 and define apices 813 (for example, outflow apices) that extend towards the outflow end 808 of the skirt frame 802. In some examples, as depicted, the struts 811 can attach to the center or midway point of each strut 810 (for example, to the free portions 816 of the struts 810) and can join an adjacent strut 811 at an apex 813. In other examples, the struts 811 can attach to other locations along the struts 810. As shown in FIG. 17, the apices 813 can at least partially define the second end 808 of the skirt frame 802 and can be axially aligned with the outflow apices 814. In some examples, as depicted, the struts 811 and the apices 813 (which can also be referred to as “floating struts” or “free struts” and “floating apices” or “free apices”, respectively) are not fixed or attached to the valve frame 502.

In some examples, as depicted, the outflow apices 813, 814 are arranged circumferentially around the skirt frame 802 in an alternating pattern, such that a free outflow apex 813 is adjacent to a fixed outflow apex 814. The outflow apices 813 can be circumferentially aligned with the inflow apices 812.

The struts of the skirt frame 802 can form and/or define a plurality of cells (that is, openings) in the skirt frame 802. For example, the struts 810 and 811 can at least partially form and/or define a plurality of cells 818 that are circumferentially spaced apart around the skirt frame 802. Specifically, each cell 818 can be formed by segments of two struts 810a, 810b of the first row of struts 810 and the entire length of two struts 811a, 811b of the second row of struts 811. Each cell 818 can have a diamond shape including the apices 812 at or near the first end 806 of the skirt frame 802 and the apices 813 at or near the second end 808 of the skirt frame 802. In some examples, the skirt frame 802 comprises nine cells 818 that are circumferentially spaced apart around the skirt frame 802 in a row. However, in other examples, the skirt frame 802 can comprise a greater or fewer number of cells 818.

Similar to the skirt frame 402 and the skirt frame 702, the skirt frame 802 can be configured to radially bulge or flex away from the valve frame 502 to improve sealing of the prosthetic valve 500 against the tissue of the native valve annulus and help reduce paravalvular leakage. In particular, the intermediate portions 816 of the struts 810 (that is, the portions of the struts 810 that are not fixed or attached to the valve frame 502) and/or the free struts 811 radially bulge or flex away from the valve frame 502 when the prosthetic heart valve 500 is radially expanded. Specifically, during radial expansion, the valve frame 502 and the skirt frame 802 foreshorten along their respective axial lengths, causing the free portions of the skirt frame 802 (for example, the free portions 816 and/or the free struts 811, etc.) to radially bulge or flex away from the valve frame 502 by a flex amount, adding to the overall radial profile of the prosthetic heart valve 500 when radially expanded.

In some examples, the skirt frame 802 can be relatively thin, such that the skirt assembly 800 does not significantly add to the overall outer diameter of the valve frame 502 when the prosthetic heart valve 500 is in a crimped or radially compressed state (such as for delivery). For example, the thickness of the struts 810, 811 can be 0.08 mm or less, and preferably 0.02 mm to 0.05 mm. In this way, the skirt frame 802 advantageously preserves the crimped profile of the valve frame 502, while also bulging radially outward when the valve frame 502 is in the radially expanded state to improve sealing against the native tissue upon implantation and reduce paravalvular leakage.

As described above, the skirt 804 can be coupled to the skirt frame 802. Accordingly, when the skirt frame 802 radially bulges or flexes away from the valve frame 502, the skirt 804 is also bulged or flexed outwardly in the radial direction. The radial bulging or flexing of the skirt 804 can help improve the sealing of the prosthetic heart valve 500 against the tissue of the native valve annulus and help reduce paravalvular leakage past the prosthetic valve.

In some examples, one or more of struts 810, 811 can be twisted similar to struts 710 of skirt frame 702.

FIG. 18 illustrates another example of a skirt assembly 900 that is coupled to a prosthetic heart valve (for example, prosthetic heart valve 100 of FIG. 1A, prosthetic heart valve 500 of FIG. 11 (for example, in lieu of outer skirt 506), and/or any of prosthetic heart valve described herein). In particular, as depicted, the skirt assembly 900 is coupled to valve frame 502 of the prosthetic heart valve 500. The skirt assembly 900 can function as a scaling mechanism for the prosthetic valve 500 by sealing against the tissue of the native valve annulus and helping to reduce paravalvular leakage past the prosthetic valve 500. For example, as shown in FIG. 18, the skirt assembly 900 may include a skirt frame 902 (also referred to as a sealing frame) and a skirt 904 (also referred to as a sealing member) mounted around an outer surface of the valve frame 502.

As shown in FIG. 18, the skirt assembly 900 may include a skirt 904 that is coupled to the skirt frame 902. For example, the skirt 904 can be coupled to the skirt frame 902 in any manner described above in connection with coupling the skirt 404 to the skirt frame 402. Similar to the skirt 404, the skirt 904 can function as a sealing member for the prosthetic valve 500 by sealing against the tissue of the native valve annulus and helping to reduce paravalvular leakage past the prosthetic valve 500. As will be described in more detail below, the skirt frame 902 can be configured to radially bulge or flex away from the valve frame 502 to improve sealing of the prosthetic valve 500 against the tissue of the native valve annulus and help reduce paravalvular leakage.

The skirt frame 902 can be coupled (for example, fixedly mounted) to an outer surface of the valve frame 502. For example, the skirt frame 902 can be coupled to the valve frame 502 in manners similar to those described above.

The skirt frame 902 can be made of any of various suitable plastically expandable materials (for example, stainless steel, etc.) or self-expanding materials (for example, Nitinol), similar to skirt frame 402 as described above.

The skirt frame 902 can be mounted to any number of struts of the valve frame 502. In some examples, as depicted, portions of the skirt frame 902 can be fixed to each of the angled struts 530, 532, 534 (see FIG. 13) as well as to the axial struts 538, 540. As shown, the skirt frame 902 is positioned towards the inflow end 508 of the valve frame 502. In particular, a first (or inflow) end 906 of the skirt frame 902 is coupled to the angled struts 530 (for example, at the inflow end 508) and a second (or outflow) end 908 of the skirt frame 902 is coupled to the inflow end portions 546 of the axial struts 540 and inflow end portions of the axial struts 538 (for example, between inflow and outflow ends 508, 510 of the valve frame 502). For example, the second end 908 of the skirt frame 902 is coupled to the valve frame 502 at a location where struts 534 of the cells 518 connect to the axial struts 538, 540 (for example, the second end 908 of the skirt frame 902 is positioned closer to the inflow end 508 than the commissure windows 542, etc.). In some examples, the skirt frame 902 can also be coupled to the angled struts 532, 534.

The skirt frame 902 can include a plurality of circumferentially extending, interconnected struts 910. As illustrated in FIG. 18, the struts 910 of the skirt frame 902 are generally aligned with the angled struts 530, 532, 534 of the valve frame 502 in a radially expanded state. Specifically, the interconnected struts 910 can include a plurality of angled struts 930, 932, and 934 arranged in a plurality of rows of circumferentially extending rows of angled struts, with the rows being arrayed along the length of the skirt frame 902 between the outflow end 908 and the inflow end 906. For example, the skirt frame 902 can comprise a first row of angled struts 930 arranged end-to-end and extending circumferentially at the inflow end 906 of the skirt frame 902 (for example, aligned with angled struts 530); a second row of circumferentially extending, angled struts 932 (for example, aligned with angled struts 532); and a third row of circumferentially extending, angled struts 934 at the outflow end 908 of the skirt frame 902 (for example, aligned with angled struts 534).

The struts 930, 932, 934 of the skirt frame 902 can form and/or define a plurality of cells (that is, openings) in the skirt frame 902. For example, the struts 930, 932, 934 can at least partially form and/or define multiple rows of open cells between the inflow end 906 and the outflow end 908 of the skirt frame 902 (for example, corresponding to the cells 526, 528 of the valve frame 502). Specifically, the struts 930 and 932 can define a first row of cells 918 (for example, aligned with cells 528 of the valve frame 502) and the struts 932 and 934 can define a second row of cells 920 (for example, aligned with cells 526 of the valve frame 502). As shown, the first cells 918 are adjacent to the second cells 920 and the struts 932 partially defines both the first and second cells 918, 920.

In some examples, as shown in FIG. 18, each row of cells 918, 920 comprises nine cells. In alternate examples, the skirt frame 902 can comprise more than two rows of cells (for example, three, four or five) and/or more or less than nine cells per row.

While the struts 910 of the skirt assembly 900 are shown in this example as corresponding to and/or aligning with selected struts of the valve frame 502 (for example, struts 530, 532, 534 located towards the inflow end 508 of the valve frame 502), in other examples, struts of a skirt assembly can correspond and/or align with struts of any other radially expandable frame for a prosthetic heart valve (for example, any valve frame described herein). In this way, cells of a skirt frame can align with cells of a valve frame.

Each first cell 918 can have a diamond shape including first and second apices 922. Each second cell 920 can have a diamond shape including first and second apices 924. In some examples, as shown in FIG. 18, the skirt frame 902 includes axially-extending struts 926 (which can also be referred to herein as “axial struts”) that extend in an axial direction (relative to the central longitudinal axis 522) and interconnect with the struts 910. In particular, the axial struts 926 extend axially between apices 922 of each first cell 918 and apices 924 of each second cell 920. As shown, the skirt frame 902 comprises an equal number of axial struts 926 and cells 918, 920, such that one axial strut 926 can be coupled to each cell 918, 920 (for example, eighteen axial struts 926 corresponding to nine first cells 918 and nine second cells 920, etc.). In other examples, an axial strut 926 can be coupled to fewer than all of the cells 918, 920 (for example, only cells 918, only cells 920, alternating cells 918, 920, etc.).

In some examples, the struts 910 of the skirt frame 902 are fixedly coupled to the valve frame 502 and the axial struts 926 are not fixed to the valve frame 502, such that the axial struts 926 are permitted to flex or bulge radially outward from the struts 910 and the valve frame 502 when the valve frame 502 (and therefore the prosthetic heart valve 500 and the skirt assembly 900) is in a radially expanded state, while the struts 910 do not budge outwardly away from the valve frame. Accordingly, in some examples, the struts 910 can also be referred to as “fixed struts” and the axial struts 926 can also be referred to as “free struts” or “flex struts.”

In other examples, only a portion of the struts 910 are fixedly coupled to the valve frame 502, rather than the entirety of each strut 910. For example, only the apices 922, 924 that are located at the first and second ends 906, 908 of the skirt frame 902 can be fixedly coupled to the valve frame 502 in some examples. As another example, selected ones of apices 922, 924 (for example, in a zig-zag pattern, all of the apices 922, 924, etc.) can be fixedly coupled to the valve frame 502, with the remaining portions of the skirt frame 902 being free relative to the valve frame 502. In such examples, the portions of the struts 910 between the locations where they fixed relative to the valve frame can bulge outwardly relative to the valve frame when the valve frame is radially expanded.

Similar to the skirt frames 402, 702, 802, the skirt frame 902 can be configured to radially bulge or flex away from the valve frame 502 to improve sealing of the prosthetic valve 500 against the tissue of the native valve annulus and help reduce paravalvular leakage. In particular, the free axial struts 926 (and in some examples, portions of the struts 910 that are not fixed or attached to the valve frame 502) are configured to radially bulge or flex away from the valve frame 502 when the prosthetic heart valve 500 is radially expanded. Specifically, during radial expansion, the valve frame 502 and the skirt frame 902 foreshorten along their respective axial lengths and the free portions of the skirt frame 902 (for example, at least the axial struts 926, etc.) are permitted to radially bulge or flex away from the valve frame 502 by a flex amount, adding to the overall radial profile of the prosthetic heart valve 500 when radially expanded.

In some examples, the skirt frame 902 can be relatively thin, such that the skirt assembly 900 does not significantly add to the overall outer diameter of the valve frame 502 when the prosthetic heart valve 500 is in a crimped or radially compressed state (such as for delivery). For example, the thickness of the struts 910 and the axial struts 926 can be 0.08 mm or less, and preferably 0.02 mm to 0.05 mm. In this way, the skirt frame 902 advantageously preserves the crimped profile of the valve frame 502, while also bulging radially outward when the valve frame 502 is in the radially expanded state to improve sealing against the native tissue upon implantation and reduce paravalvular leakage.

As described above, the skirt 904 can be coupled to the skirt frame 902. Accordingly, when the skirt frame 902 radially bulges or flexes away from the valve frame 502, the skirt 904 is also bulged or flexed outwardly in the radial direction. The radial bulging or flexing of the skirt 904 can help improve the sealing of the prosthetic heart valve 500 against the tissue of the native valve annulus and help reduce paravalvular leakage past the prosthetic valve.

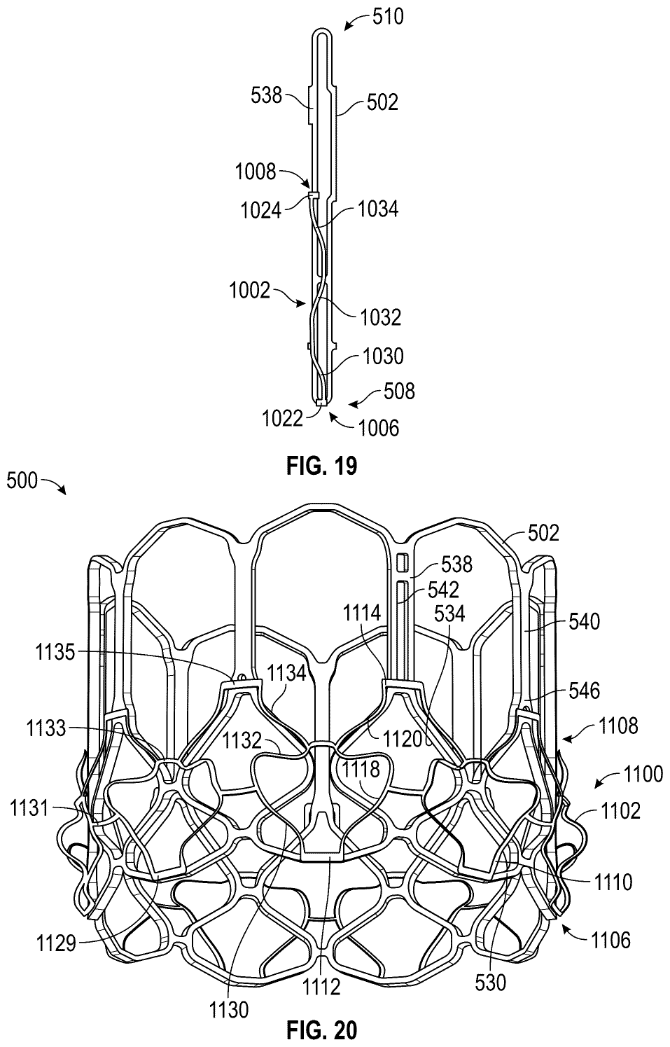

FIG. 19 illustrates a portion of an example skirt frame 1002 (also referred to as a sealing frame) having three rows of angled struts (for example, similar to skirt frame 902, etc.) that is coupled to the valve frame 502 in a radially compressed state. Specifically, FIG. 19 illustrates only one strut per row of the skirt frame 1002 and only one cell column of the valve frame 502. Although only one strut is shown per row, the skirt frame 1002 can include a plurality of angled struts 1030, 1032, and 1034 arranged in a plurality of rows of circumferentially extending rows of angled struts, similar to skirt frame 902. For example, the skirt frame 1002 can comprise a first row of angled struts 1030 arranged end-to-end and extending circumferentially at an inflow end 1006 of the skirt frame 1002; a second row of circumferentially extending, angled struts 1032; and a third row of circumferentially extending, angled struts 1034 at the outflow end 1008 of the skirt frame 1002. As shown, the skirt frame 1002 includes an inflow apex 1022 (for example, defined by struts 1030) coupled to the inflow end 508 of the valve frame 502 and an outflow apex 1024 (for example, defined by struts 1034) coupled to the axial strut 538. The inflow apex 1022 is circumferentially offset from the outflow apex 1024. It should be appreciated that the skirt frame 1002 is configured similarly to skirt frame 902, although the skirt frame 1002 does not include axial struts (for example, such as axial struts 926).

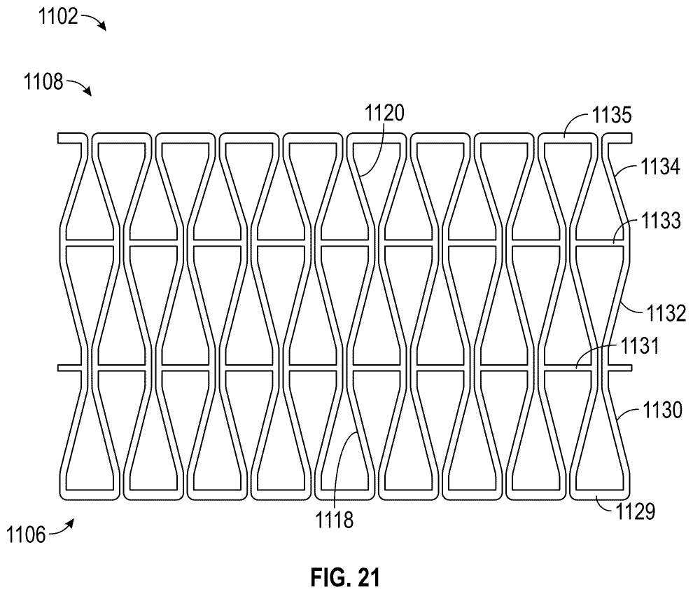

FIGS. 20-21 illustrate another example of a skirt assembly 1100 that is coupled to a prosthetic heart valve (for example, prosthetic heart valve 100 of FIG. 1A, prosthetic heart valve 500 of FIG. 11 (for example, in lieu of outer skirt 506), and/or any of prosthetic heart valve described herein). In particular, as depicted, the skirt assembly 1100 is coupled to valve frame 502 of the prosthetic heart valve 500. The skirt assembly 1100 (also referred to as a sealing assembly) can function as a sealing mechanism for the prosthetic valve 500 by scaling against the tissue of the native valve annulus and helping to reduce paravalvular leakage past the prosthetic valve 500. For example, the skirt assembly 1100 may include a skirt frame 1102 (also referred to as a sealing frame) and a skirt or sealing member or sealing layer (not shown) (for example, similar to skirts 404, 704, 804, 904) mounted around an outer surface of the valve frame 502. FIG. 20 shows the skirt assembly 1100 and the valve frame 502 in a radially expanded state. FIG. 21 shows the skirt frame 1102 by itself in a flattened state before the skirt frame 1102 is subjected to any shape setting.

The skirt frame 1102 can be coupled (for example, fixedly mounted) to an outer surface of the valve frame 502. For example, the skirt frame 1102 can be coupled to the valve frame 502 in manners similar to those described above.

The skirt frame 1102 can be made of any of various suitable plastically expandable materials (for example, stainless steel, etc.) or self-expanding materials (for example, Nitinol), similar to skirt frame 402 as described above.

The skirt frame 1102 can be mounted to any number of struts of the valve frame 502. In some examples, as depicted, portions of the skirt frame 1102 can be fixed to the angled struts 530 as well as to the axial struts 538, 540. As shown, the skirt frame 1102 is positioned towards the inflow end 508 of the valve frame 502. In particular, a first (or inflow) end 1106 of the skirt frame 1102 is coupled to the angled struts 530 (for example, at the inflow end 508) and a second (or outflow) end 1108 of the skirt frame 1102 is coupled to the inflow end portions 546 of the axial struts 540 and inflow end portions of the axial struts 538 (for example, between inflow and outflow ends 508, 510 of the valve frame 502). For example, as depicted, the second end 1108 of the skirt frame 1102 is coupled to the valve frame 502 at a location where struts 534 of the cells 518 connect to the axial struts 538, 540 (for example, the second end 1108 of the skirt frame 1102 is positioned closer to the inflow end 508 than the commissure windows 542, etc.).

The skirt frame 1102 can include a plurality of circumferentially extending, interconnected struts 1110. As illustrated in FIGS. 20-21, the struts 1110 can include a plurality of angled struts 1130, 1132, and 1134 and a plurality of circumferentially-extending or flat struts 1129, 1131, 1133, and 1135. The struts 1129, 1131, 1133, and 1135 interconnect adjacent ends of adjacent angled struts. The struts 1110 can be arranged in a plurality of rows of angled and flat struts, with the rows being arrayed along the length of the skirt frame 1102 between the outflow end 1108 and the inflow end 1106. For example, the skirt frame 1102 can comprise a first row of struts including flat struts 1129, angled struts 1130 and flat struts 1131 arranged end-to-end and extending circumferentially at the inflow end 1106 of the skirt frame 1102. Specifically, the first row alternates between flat struts (either struts 1129 or 1131) and angled struts 1130. The skirt frame 1102 can comprise a second row of struts including flat struts 1131, angled struts 1132, and flat struts 1133 arranged end-to-end and extending circumferentially the skirt frame 1102 between the inflow end 1106 and the outflow end 1108 of the skirt frame 1102. Specifically, the second row alternates between flat struts (either struts 1131 or 1133) and angled struts 1132. The skirt frame 1102 can comprise a third row of circumferentially extending struts including flat struts 1133, angled struts 1134, and flat struts 1135 at the outflow end 1108 of the skirt frame 1102. Specifically the third row alternates between flat struts (either struts 1133 or 1135) and angled struts 1134.

The struts 1129-1135 of the skirt frame 1102 can form and/or define a plurality of cells (that is, openings) in the skirt frame 1102. For example, the struts 1129-1135 can at least partially form and/or define multiple rows of open cells between the inflow end 1106 and the outflow end 1108 of the skirt frame 1102. Specifically, the struts 1129, 1130, 1132, and 1133 can define a first row of cells 1118 and the struts 1131, 1132, 1134, and 1135 can define a second row of cells 1120. As shown, the first cells 1118 are circumferentially spaced apart by flat struts 1131 and the second cells 1120 are circumferentially spaced apart by flat struts 1133. As such, the flat struts 1131, 1133 can serve as junctions between the first and second cells 1118, 1120, respectively.

The struts 1129 can define a plurality of inflow apices 1112 at the first end 1106 of the skirt frame 1102 and the struts 1135 can define a plurality of outflow apices 1114 at the second end 1108 of the skirt frame 1102. The inflow and outflow apices 1112, 1114 are flat or circumferentially extending. In some examples, as depicted, each of the inflow and outflow apices 1112, 1114 are coupled to the valve frame 502 and can be referred to as “fixed” inflow and outflow apices 1112, 1114. In other examples, only some of the inflow and outflow apices 1112, 1114 are coupled to the valve frame 502 (for example, at least one fixed inflow apex 1112 and at least one fixed outflow apex 1114, etc.).

In some examples, as shown in FIGS. 20-21, each row of cells 1118, 1120 comprises nine cells. In alternate examples, the skirt frame 1102 can comprise more than two rows of cells (for example, three, four or five) and/or more or less than nine cells per row.

In some examples, the struts 1129, 1135 of the skirt frame 1102 are fixedly coupled to the valve frame 502 and the other struts 1130-1134 are not fixed to the valve frame 502, such that the struts 1130-1134 are permitted to flex or bulge radially outward from the valve frame 502 when the valve frame 502 (and therefore the prosthetic heart valve 500 and the skirt assembly 900) is in a radially expanded state. Accordingly, in some examples, the struts 1129, 1135 can also be referred to as “fixed struts” and the struts 1130-1134 can also be referred to as “free struts” or “flex struts.”

Similar to the skirt frames 402, 702, 802, 902, the skirt frame 1102 can be configured to radially bulge or flex away from the valve frame 502 to improve sealing of the prosthetic valve 500 against the tissue of the native valve annulus and help reduce paravalvular leakage. In particular, the free struts 1130-1134 are configured to radially bulge or flex away from the valve frame 502 when the prosthetic heart valve 500 is radially expanded. Specifically, during radial expansion, the valve frame 502 and the skirt frame 1102 foreshorten along their respective axial lengths and the free portions of the skirt frame 1102 (for example, struts 1130-1134, etc.) are permitted to radially bulge or flex away from the valve frame 502 by a flex amount, adding to the overall radial profile of the prosthetic heart valve 500 when radially expanded.

In some examples, the skirt frame 1102 can be relatively thin, such that the skirt assembly 1100 does not significantly add to the overall outer diameter of the valve frame 502 when the prosthetic heart valve 500 is in a crimped or radially compressed state (such as for delivery). For example, the thickness of the struts 1110 can be 0.08 mm or less, and preferably 0.02 mm to 0.05 mm. In this way, the skirt frame 902 advantageously preserves the crimped profile of the valve frame 502, while also bulging radially outward when the valve frame 502 is in the radially expanded state to improve sealing against the native tissue upon implantation and reduce paravalvular leakage.

As described above, the skirt 1104 can be coupled to the skirt frame 1102. Accordingly, when the skirt frame 1102 radially bulges or flexes away from the valve frame 502, the skirt 1104 is also bulged or flexed outwardly in the radial direction. The radial bulging or flexing of the skirt 1104 can help improve the sealing of the prosthetic heart valve 500 against the tissue of the native valve annulus and help reduce paravalvular leakage past the prosthetic valve.

Any of the sealing members disclosed herein (for example, sealing members 404, 704, 804, 904) can be formed from a variety of suitable materials and can have various constructions. For example, the sealing members can be formed from a textile material, such as a braided, woven, knitted fabric or a non-woven fabric (for example, a felt) and can comprise synthetic yarns or fibers made of any of various polymers, including, without limitation, PET, ePTFE, PTFE, TPU, UHMWPE, PEEK, or PE. Alternatively, the scaling members can comprise a non-fibrous membrane or sheet of material made of any of various polymers, including, without limitation, PET, ePTFE, PTFE, TPU, UHMWPE, PEEK, or PE. In other examples, the sealing members can comprise natural tissue, such as pericardial tissue (for example, bovine pericardial tissue or pericardial tissue from other animals).

It is to be understood that the skirt assemblies (for example, skirt assemblies 400, 700, 800, 900, 1100) shown and described herein are merely exemplary and that other skirt assemblies are within the scope of the present disclosure. For example, skirt frames of skirt assemblies can include other arrangements of struts or other members that bulge or flex radially away from a valve frame. Further, any of the disclosed skirt assemblies (for example, skirt assemblies 400, 700, 800, 900, 1100) can be coupled to any type of prosthetic transcatheter heart valve, including self-expandable prosthetic heart valves, balloon-expandable prosthetic heart valves and mechanically-expandable prosthetic heart valves. Advantageously, the skirt assemblies of the present disclosure preserve the crimped profile of the prosthetic valve, while bulging radially away therefrom during expansion to improve sealing against the native tissue upon implantation. Furthermore, the skirt frames described herein allow the skirt assemblies to expand further away from the valve frame in a controlled manner, such that the desired profile, size and locations of the bulging portions can be predetermined.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (e.g., with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a valve frame comprising an outflow end and an inflow end, wherein the valve frame is radially expandable from a radially compressed state to a radially expanded state; a plurality of leaflets disposed within and coupled to the valve frame; and a skirt assembly mounted to an outer surface of the valve frame, wherein the skirt assembly comprises a sealing layer and a skirt frame including a plurality of interconnected struts, wherein the skirt frame has a plurality of inflow apices and outflow apices, wherein selected ones of the inflow apices are fixed to the valve frame and selected ones of the outflow apices are fixed to the valve frame, wherein one or more of the struts are configured to flex in an outward radial direction when the valve frame is in the radially expanded state to cause the sealing layer to protrude outwardly from the valve frame.

Example 2. The prosthetic heart valve of any example herein, particularly example 1, wherein the sealing layer is radially outside of the skirt frame.

Example 3. The prosthetic heart valve of any example herein, particularly either example 1 or example 2, wherein the skirt frame comprises a shape-memory material.

Example 4. The prosthetic heart valve of any example herein, particularly example 3, wherein the one or more of the struts of the skirt frame are configured to flex in the outward radial direction relative to the valve frame independent of a degree of radial expansion of the valve frame.

Example 5. The prosthetic heart valve of any example herein, particularly example 3, wherein when the valve frame is in the radially compressed state, the skirt frame is in a radially compressed state, wherein the compressed state is a shape-memory state of the skirt frame, and wherein radially expanding the valve frame results in deformation of the skirt frame from the shape-memory state to a deformed state.

Example 6. The prosthetic heart valve of any example herein, particularly any one of examples 1-5, wherein every other inflow apex is fixed to the valve frame.

Example 7. The prosthetic heart valve of any example herein, particularly any one of examples 1-5, wherein all of the inflow apices are fixed to the valve frame.

Example 8. The prosthetic heart valve of any example herein, particularly any one of examples 1-7, wherein every other outflow apex is fixed to the valve frame.

Example 9. The prosthetic heart valve of any one of examples 1-7, wherein all of the outflow apices are fixed to the valve frame.

Example 10. The prosthetic heart valve of any example herein, particularly any one of examples 1-9, wherein the inflow apices are circumferentially offset from the outflow apices.

Example 11. The prosthetic heart valve of any example herein, particularly any one of examples 1-10, wherein the inflow apices and the outflow apices are arranged in an alternating pattern.

Example 12. The prosthetic heart valve of any example herein, particularly any one of examples 1-11, wherein the skirt frame comprises multiple rows of angled struts.

Example 13. The prosthetic heart valve of any example herein, particularly example 12, wherein a first row of angled struts defines the inflow apices, and wherein a second row of angled struts defines the outflow apices.

Example 14. The prosthetic heart valve of any example herein, particularly example 12, wherein a first row of angled struts defines the inflow apices and a first set of the outflow apices, and wherein a second row of angled struts defines a second set of the outflow apices.

Example 15. The prosthetic heart valve of any example herein, particularly example 14, wherein struts of the first row are longer than struts of the second row.

Example 16. The prosthetic heart valve of any example herein, particularly either example 14 or example 15, wherein the first set of the outflow apices are fixed to the valve frame and wherein the second set of the outflow apices are free relative to the valve frame.

Example 17. The prosthetic heart valve of any example herein, particularly any one of examples 12-16, wherein the multiple rows of angled struts form at least one row of cells.

Example 18. The prosthetic heart valve of any example herein, particularly example 17, wherein each cell comprises at least one fixed apex that is fixed to the valve frame.

Example 19. The prosthetic heart valve of any example herein, particularly example 18, wherein each cell comprises at least one free apex that is moveable relative to the valve frame.

Example 20. The prosthetic heart valve of any example herein, particularly any one of examples 1-11, wherein the skirt frame comprises a single row of angled struts.

Example 21. The prosthetic heart valve of any example herein, particularly example 20, wherein each strut in the single row of angled struts is twisted.

Example 22. The prosthetic heart valve of any example herein, particularly example 21, wherein a width of a strut is greater than a thickness of the strut.

Example 23. The prosthetic heart valve of any example herein, particularly any one of examples 1-22, wherein the valve frame is mechanically expandable.

Example 24. The prosthetic heart valve of any example herein, particularly any one of examples 1-23, wherein a thickness of the skirt frame is 0.08 mm or less.

Example 25. The prosthetic heart valve of any example herein, particularly example 24, wherein the thickness of the skirt frame is within a range of about 0.02 mm to about 0.05 mm.

Example 26. The prosthetic heart valve of any example herein, particularly any one of examples 1-25, wherein the skirt frame comprises a plurality of angled struts and a plurality of axially extending struts.

Example 27. A prosthetic heart valve comprising: a valve frame being radially expandable and compressible between a radially compressed state and a radially expanded state; a plurality of leaflets disposed within and coupled to the valve frame; and a skirt assembly mounted to an outer surface of the valve frame, wherein the skirt assembly comprises a scaling layer and a skirt frame, wherein the skirt frame comprises a plurality of interconnected struts forming at least one row of cells, wherein radially expanding the valve frame from the radially compressed state to the radially expanded state results in the skirt assembly flexing radially outwardly relative to the valve frame.

Example 28. The prosthetic heart valve of any example herein, particularly example 27, wherein the sealing layer comprises a fabric skirt.

Example 29. The prosthetic heart valve of any example herein, particularly either example 27 or example 28, wherein the skirt frame is embedded within the sealing layer.

Example 30. The prosthetic heart valve of any example herein, particularly any one of examples 27-29, wherein the skirt frame comprises fixed apices coupled to the valve frame and free apices moveable relative to the valve frame.

Example 31. The prosthetic heart valve of any example herein, particularly example 30, wherein at least one of the fixed apices is disposed at an inflow end of the skirt frame, and wherein at least one of the fixed apices is disposed at an outflow end of the skirt frame.

Example 32. The prosthetic heart valve of any example herein, particularly either example 30 or example 31, wherein each cell comprises one fixed apex and one free apex.

Example 33. The prosthetic heart valve of any example herein, particularly any one of examples 30-32, wherein the fixed apices are circumferentially spaced apart.

Example 34. The prosthetic heart valve of any example herein, particularly any one of examples 27-33, wherein cells of a row of cells are circumferentially spaced apart.

Example 35. The prosthetic heart valve of any example herein, particularly any one of examples 27-33, wherein the skirt frame comprises multiple rows of cells.

Example 36. The prosthetic heart valve of any example herein, particularly any one of examples 27-35, wherein the valve frame comprises a plurality of interconnected struts forming at least one row of cells, wherein a row of cells of the valve frame includes the same number of cells as a row of cells as the skirt frame.

Example 37. The prosthetic heart valve of any example herein, particularly any one of examples 27-36, wherein the skirt frame comprises a plurality of axially extending struts interconnected with the cells of the skirt frame.

Example 38. A prosthetic heart valve comprising: a valve frame comprising an outflow end and an inflow end, wherein the valve frame is radially expandable from a radially compressed state to a radially expanded state; a plurality of leaflets disposed within and coupled to the valve frame; and a sealing assembly mounted to an outer surface of the valve frame, the sealing assembly comprising a sealing member and a sealing frame, wherein the sealing frame comprises a shape-memory material, wherein the sealing frame is in a radially compressed state when the valve frame is in the radially compressed state, wherein the compressed state of the sealing frame is a shape-memory state, and wherein radially expansion of the valve frame results in deformation of the sealing frame from the shape-memory state to a deformed state.

Example 39. The prosthetic heart valve of any example herein, particularly example 38, wherein the sealing frame bulges radially outward from the valve frame in the deformed state.

Example 40. The prosthetic heart valve of any example herein, particularly either example 38 or example 39, wherein the sealing frame comprises a plurality of interconnected angled struts.

Example 41. The prosthetic heart valve of any example herein, particularly example 40, wherein the sealing frame comprises inflow apices and outflow apices.

Example 42. The prosthetic heart valve of any example herein, particularly example 41, wherein selected ones of the inflow apices are coupled to the inflow end of the valve frame, and wherein selected ones of the outflow apices are coupled to the valve frame at an intermediate location between the inflow and outflow ends of the valve frame.

Example 43. The prosthetic heart valve of any example herein, particularly example 42, wherein all of the inflow and outflow apices are coupled to the valve frame.

Example 44. The prosthetic heart valve of any example herein, particularly example 42, wherein every other inflow apex is coupled to the valve frame, and wherein every other outflow apex is coupled to the valve frame.

Example 45. The prosthetic heart valve of any example herein, particularly any one of examples 40-44, wherein the struts define at least one row of cells.

Example 46. The prosthetic heart valve of any example herein, particularly example 45, wherein cells of a row of cells are circumferentially spaced apart.

Example 47. The prosthetic heart valve of any example herein, particularly example 46, wherein the sealing frame comprises circumferentially extending struts, wherein a circumferentially extending strut is coupled to a pair of adjacent cells in the row of cells.

Example 48. The prosthetic heart valve of any example herein, particularly example 45, wherein the sealing frame comprises a first row of cells axially that is aligned with a first row of cells of the valve frame.

Example 49. The prosthetic heart valve of any example herein, particularly example 48, wherein the sealing frame comprises axially extending struts, wherein an axially extending strut is coupled to apices of a cell in the first row of cells of the sealing frame.

Example 50. The prosthetic heart valve of any example herein, particularly either example 48 or example 49, wherein the sealing frame comprises a second row of cells that is axially aligned with a second row of cells of the valve frame.

Example 51. The prosthetic heart valve of any example herein, particularly example 50, wherein the first row of cells of the sealing frame is adjacent to the second row of cells of the sealing frame.

Example 52. A delivery apparatus comprising: a delivery device; and a prosthetic valve releasably coupled to the delivery device, the prosthetic valve comprising a valve frame that is expandable between a radially compressed state and a radially expanded state, a valvular structure mounted within the valve frame, and a sealing assembly mounted to an outer surface of the valve frame, wherein the sealing assembly comprises a sealing layer and a sealing frame including a plurality of interconnected struts, wherein the sealing frame has a plurality of inflow apices and outflow apices, wherein selected ones of the inflow apices are fixed to the valve frame and selected ones of the outflow apices are fixed to the valve frame, wherein one or more of the struts are configured to flex in an outward radial direction when the valve frame is in the radially expanded state to cause the sealing layer to protrude outwardly from the sealing frame.

Example 53. The delivery apparatus of any example herein, particularly example 52, wherein the sealing layer is radially outside of the sealing frame.

Example 54. The delivery apparatus of any example herein, particularly either example 52 or example 53, wherein the sealing frame comprises a shape-memory material.

Example 55. The prosthetic heart valve of any example herein, particularly example 54, wherein the one or more of the struts of the sealing frame are configured to flex in the outward radial direction relative to the valve frame independent of a degree of radial expansion of the valve frame.

Example 56. The delivery apparatus of any example herein, particularly example 54, wherein when the valve frame is in the radially compressed state, the sealing frame is in a radially compressed state, wherein the compressed state is a shape-memory state of the sealing frame, and wherein radially expanding the valve frame results in deformation of the sealing frame from the shape-memory state to a deformed state.

Example 57. The delivery apparatus of any example herein, particularly any one of examples 52-56, wherein every other inflow apex is fixed to the valve frame.

Example 58. The delivery apparatus of any example herein, particularly any one of examples 52-56, wherein all of the inflow apices are fixed to the valve frame.

Example 59. The delivery apparatus of any example herein, particularly any one of examples 52-58, wherein every other outflow apex is fixed to the valve frame.

Example 60. The delivery apparatus of any example herein, particularly any one of examples 52-58, wherein all of the outflow apices are fixed to the valve frame.

Example 61. The delivery apparatus of any example herein, particularly any one of examples 52-60, wherein the inflow apices are circumferentially offset from the outflow apices.

Example 62. The delivery apparatus of any example herein, particularly any one of examples 52-61, wherein the inflow apices and the outflow apices are arranged in an alternating pattern.

Example 63. The delivery apparatus of any example herein, particularly any one of examples 52-62, wherein the sealing frame comprises multiple rows of angled struts.

Example 64. The delivery apparatus of any example herein, particularly example 63, wherein a first row of angled struts defines the inflow apices, and wherein a second row of angled struts defines the outflow apices.

Example 65. The delivery apparatus of any example herein, particularly example 63, wherein a first row of angled struts defines the inflow apices and a first set of the outflow apices, and wherein a second row of angled struts defines a second set of the outflow apices.

Example 66. The delivery apparatus of any example herein, particularly example 65, wherein struts of the first row are longer than struts of the second row.

Example 67. The delivery apparatus of any example herein, particularly either example 65 or example 66, wherein the first set of the outflow apices are fixed to the valve frame and wherein the second set of the outflow apices are free relative to the valve frame.

Example 68. The delivery apparatus of any example herein, particularly any one of examples 63-67, wherein the multiple rows of angled struts form at least one row of cells.

Example 69. The delivery apparatus of any example herein, particularly example 68, wherein each cell comprises at least one fixed apex that is fixed to the valve frame.

Example 70. The delivery apparatus of any example herein, particularly example 69, wherein each cell comprises at least one free apex that is moveable relative to the valve frame.

Example 71. The delivery apparatus of any example herein, particularly any one of examples 52-62, wherein the sealing frame comprises a single row of angled struts.

Example 72. The delivery apparatus of any example herein, particularly example 71, wherein each strut in the single row of angled struts is twisted.

Example 73. The delivery apparatus of any example herein, particularly example 72, wherein a width of a strut is greater than a thickness of the strut.

Example 74. The delivery apparatus of any example herein, particularly any one of examples 52-73, wherein the valve frame is mechanically expandable.

Example 75. The delivery apparatus of any example herein, particularly any one of examples 52-74, wherein a thickness of the sealing frame is 0.08 mm or less.

Example 76. The delivery apparatus of any example herein, particularly example 75, wherein the thickness of the sealing frame is within a range of about 0.02 mm to about 0.05 mm.

Example 77. The delivery apparatus of any example herein, particularly any one of examples 52-76, wherein the sealing frame comprises a plurality of angled struts and a plurality of axially extending struts.

Example 78. The delivery apparatus of any example herein, particularly any one of examples 52-77, wherein the sealing layer comprises a fabric skirt.

Example 79. The delivery apparatus or prosthetic heart valve of any example herein, particularly any of examples 1-78, wherein the delivery apparatus or prosthetic heart valve is sterilized.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a valve frame comprising an outflow end and an inflow end, wherein the valve frame is radially expandable from a radially compressed state to a radially expanded state;
a plurality of leaflets disposed within and coupled to the valve frame; and
a skirt assembly mounted to an outer surface of the valve frame, wherein the skirt assembly comprises a sealing layer and a skirt frame including a plurality of interconnected struts, wherein the skirt frame has a plurality of inflow apices and outflow apices, wherein selected ones of the inflow apices are fixed to the valve frame and selected ones of the outflow apices are fixed to the valve frame, wherein one or more of the struts are configured to flex in an outward radial direction when the valve frame is in the radially expanded state to cause the sealing layer to protrude outwardly from the valve frame.

2. The prosthetic heart valve of claim 1, wherein the sealing layer is radially outside of the skirt frame.

3. The prosthetic heart valve of claim 1, wherein the skirt frame comprises a shape-memory material.

4. The prosthetic heart valve of claim 3, wherein when the valve frame is in the radially compressed state, the skirt frame is in a radially compressed state, wherein the compressed state is a shape-memory state of the skirt frame, and wherein radially expanding the valve frame results in deformation of the skirt frame from the shape-memory state to a deformed state.

5. The prosthetic heart valve of claim 1, wherein the skirt frame comprises multiple rows of angled struts.

6. The prosthetic heart valve of claim 1, wherein the skirt frame comprises a single row of angled struts.

7. The prosthetic heart valve of claim 1, wherein the skirt frame comprises a plurality of angled struts and a plurality of axially extending struts.

8. A prosthetic heart valve comprising:
a valve frame being radially expandable and compressible between a radially compressed state and a radially expanded state;
a plurality of leaflets disposed within and coupled to the valve frame; and
a skirt assembly mounted to an outer surface of the valve frame, wherein the skirt assembly comprises a sealing layer and a skirt frame, wherein the skirt frame comprises a plurality of interconnected struts forming at least one row of cells, wherein radially expanding the valve frame from the radially compressed state to the radially expanded state results in the skirt assembly flexing radially outwardly relative to the valve frame.

9. The prosthetic heart valve of claim 8, wherein the skirt frame is embedded within the sealing layer.

10. The prosthetic heart valve of claim 8, wherein the skirt frame comprises fixed apices coupled to the valve frame and free apices moveable relative to the valve frame.

11. The prosthetic heart valve of claim 10, wherein at least one of the fixed apices is disposed at an inflow end of the skirt frame, and wherein at least one of the fixed apices is disposed at an outflow end of the skirt frame.

12. The prosthetic heart valve of claim 10, wherein each cell comprises one fixed apex and one free apex.

13. The prosthetic heart valve of claim 10, wherein the fixed apices are circumferentially spaced apart.

14. The prosthetic heart valve of claim 8, wherein cells of a row of cells are circumferentially spaced apart.

15. The prosthetic heart valve of claim 8, wherein the skirt frame comprises a plurality of axially extending struts interconnected with the cells of the skirt frame.

16. A prosthetic heart valve comprising:
a valve frame comprising an outflow end and an inflow end, wherein the valve frame is radially expandable from a radially compressed state to a radially expanded state;
a plurality of leaflets disposed within and coupled to the valve frame; and
a sealing assembly mounted to an outer surface of the valve frame, the sealing assembly comprising a sealing member and a sealing frame, wherein the sealing frame comprises a shape-memory material, wherein the sealing frame is in a radially compressed state when the valve frame is in the radially compressed state, wherein the compressed state of the sealing frame is a shape-memory state, and wherein radially expansion of the valve frame results in deformation of the sealing frame from the shape-memory state to a deformed state.

17. The prosthetic heart valve of claim 16, wherein the sealing frame bulges radially outward from the valve frame in the deformed state.

18. The prosthetic heart valve of claim 16, wherein the sealing frame comprises inflow apices and outflow apices.

19. The prosthetic heart valve of claim 18, wherein selected ones of the inflow apices are coupled to the inflow end of the valve frame, and wherein selected ones of the outflow apices are coupled to the valve frame at an intermediate location between the inflow and outflow ends of the valve frame.

20. The prosthetic heart valve of claim 18, wherein the inflow and outflow apices are coupled to the valve frame.
